(19) United States
(12) Patent Application Publication
(10) Pub. No.: US 2007/0060636 A1
(43) Pub. Date: Mar. 15, 2007

(54) High enantiomeric purity dexanabinol for pharmaceutical compositions

(76) Inventors: Haim Aviv, Rehovot (IL); Raphael Bar, Rehovot (IL); Michael Schickler, Mazkeret Batya (IL); Shimon Amselem, Rehovot (IL)

Correspondence Address: WINSTON & STRAWN LLP;PATENT DEPARTMENT, 1700 K STREET, N.W., WASHINGTON, DC, 20006, US

(21) Appl. No.: 11/503,250

(22) Filed: Aug. 14, 2006

Related U.S. Application Data
(63) Continuation-in-part of application No. 10/644,687, filed on Aug. 19, 2003.

(30) Foreign Application Priority Data
Dec. 4, 2002 (IL) 153277

(51) Int. Cl. C07D 311/80; A61K 31/353

(52) U.S. Cl. 514/453; 549/391

(57) ABSTRACT

The present invention relates to a synthetic cannabinoid, dexanabinol, of enantiomeric purity in excess of 99.90%, or to a pharmaceutically acceptable salt, ester or solvate of said compound. The present invention also relates to pharmaceutical grade compositions comprising said compound of high enantiomeric purity, and uses thereof for prevention and treatment of neurological disorders, chronic degenerative diseases, CNS poisoning, cognitive impairment, inflammatory diseases or disorders, autoimmune diseases or disorders, pain, emesis, glaucoma and wasting syndromes.

### CROSS-REFERENCE TO RELATED APPLICATIONS

This application is a continuation-in-part application of U.S. patent application Ser. No. 10/644,687, filed Aug. 19, 2003.

### FIELD OF THE INVENTION

The present invention relates to a synthetic cannabinoid, dexanabinol, of high enantiomeric purity, to pharmaceutical grade compositions comprising it, and uses thereof.

### BACKGROUND OF THE INVENTION

Stereoisomers are compounds made up of the same atoms bonded by the same sequence of bonds but having different three-dimensional structures, which are not interchangeable. These three-dimensional structures are called configurations, e.g. R and S. Optically active compounds, which have one chiral atom or more, exist as two or more isomers, called enantiomers. Enantiomers are mirror images of one another and have identical physical properties, except for the fact that they rotate the plane of polarized light in opposite directions, (+) clockwise for the dextro isomer and (−) counterclockwise for the levo isomer. Likewise, they have identical chemical properties except when interacting with stereospecific compounds. When the rates at which each enantiomer reacts or interacts with another chiral compound are sufficiently different, a clear divergence in activity is observed, and many compounds that are biologically active have inactive enantiomers.

In some cases resolving a racemic mixture into the separate enantiomers will be only of academic interest, to assess the differences in activity of the purified compounds. However, in some instances one of the enantiomers is not only devoid of the biochemical activity of interest but has its own deleterious activity. In these circumstances the separation of the enantiomers has significant practical impact, especially when the compound of interest has therapeutic activity.

The first isolation, in a pure form, of Δ⁹-tetrahydrocannabinol (Δ⁹-THC), the major psychoactive constituent of cannabis, was reported by Gaoni et al. in 1964. The absolute configuration of Δ⁹-THC was established by Mechoulam et al. in 1967 and found to be of (−)-(3R,4R) stereochemistry. It was later found that the psychotropic activity of cannabinoids resides in the natural (3R,4R) series, while the opposite enantiomeric synthetic series (3S,4S) was free of these undesirable effects. In 1967 the group of Mechoulam and coworkers also achieved the synthesis of THC (for review see Mechoulam R. and Hanus L., Chem. Phys. Lip 108: 1-13, 2000). In order to exploit the therapeutic value of cannabinoids, medicinal chemists have to “neutralize” the highly undesirable psychoactive effects, for instance by preparation and selection of synthetic non-psychotropic enantiomers.

The basic route of cannabinoid synthesis involves the condensation of a monoterpenoid with a resorcinol as shown in scheme 1. The structure of the final product depends on the substituents of the initial reagents, similarly its enantiomeric purity depends on the enantiomeric purity of the reagents.

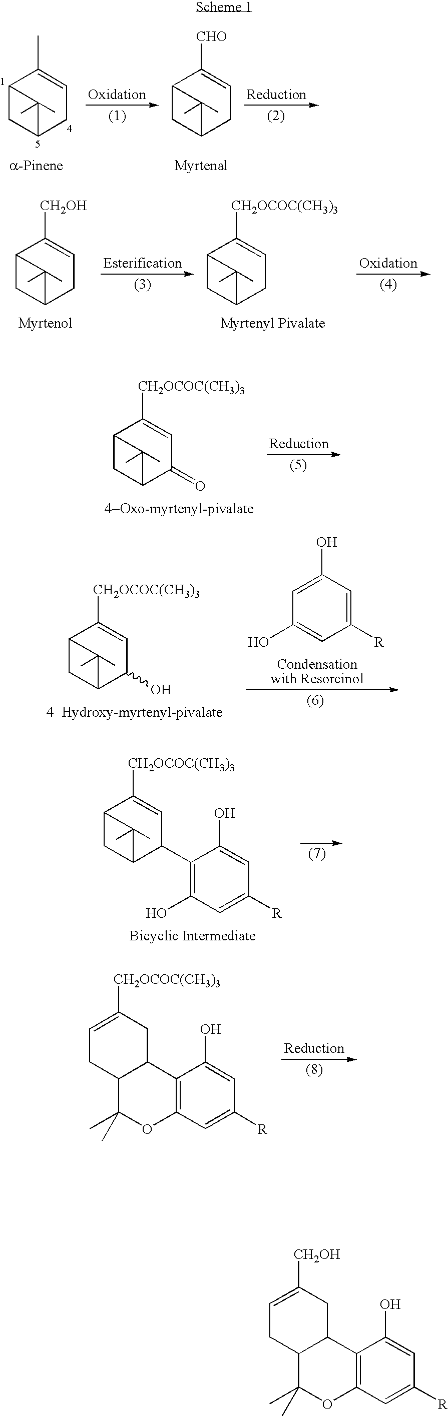

The chirality of the starting material, α-pinene, determines the chirality of the final compound. Using (+)-α-pinene will yield (1S,5R) myrtenol, and corresponding derivatives, down to classical cannabinoid analogs of the (3S,4S) configuration. Using (−)-α-pinene will yield (1R,5S) myrtenol, and corresponding derivatives, down to classical cannabinoid analogs of the (3R,4R) configuration, as shown in scheme 2. It should be noted that following the previous nomenclature, the terpenic ring was the basis for the numbering system, and the chiral centers of THC type cannabinoids were designated at carbon atoms 3 and 4. The accepted nomenclature is now based on the phenolic ring as the starting point for numbering. Thus, THC that was previously described as Δ¹-THC was later renamed Δ⁹-THC, similarly Δ⁶-THC was renamed Δ⁸-THC, and the chiral centers are at carbons 6a and 10a.

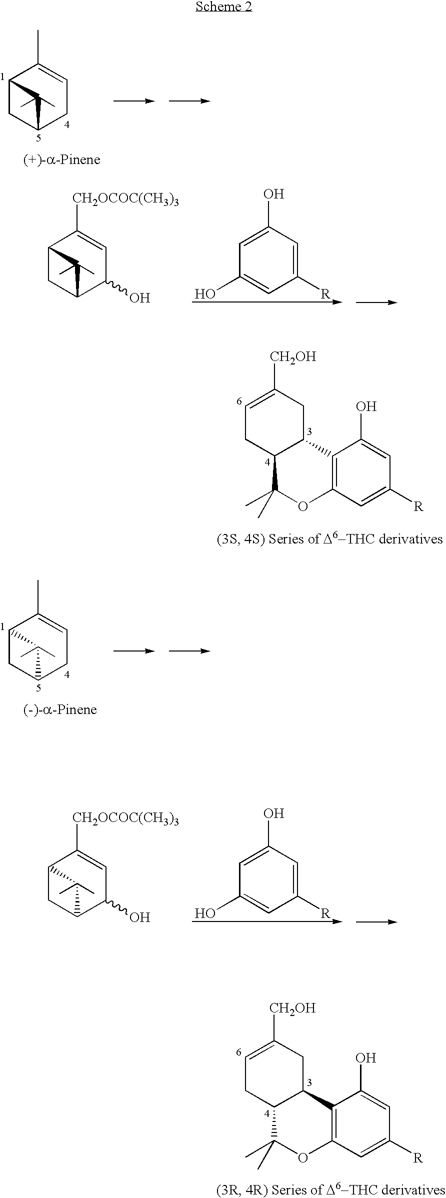

When the R substituent in schemes 1 and 2 is 1,1-dimethyl-heptyl, the compounds obtained were designated HU-210, for the (−)(3R,4R) enantiomer and HU-211, for the (+)(3S,4S) enantiomer. This pair of enantiomers was among the first to be efficiently separated, and studies performed established the fact that cannabinoid action is highly stereospecific opening the way to the search for cannabinoid receptors. HU-210 was shown to be a hundred times more psychoactive than Δ⁹-THC, the natural component of hashish, and a thousand times more psychoactive than HU-211 in a series of animal tests (Mechoulam R. et al., Tetrahedron Asymmetry 1(5): 315-8, 1990).

Beside their potent psychoactivity, cannabinoids trigger additional physiological reactions, the cardiovascular effects harboring some of the more significant consequences. In humans, the most consistent cardiovascular effects of Δ⁹-THC are peripheral vasodilatation and tachycardia. These effects manifest themselves as an increase in cardiac output, increased peripheral blood flow and variable changes in blood pressure. It has been postulated that cannabinoids induce a CNS mediated increase in sympathetic and parasympathetic nerve activity, which would result in abnormal cardiovascular outputs. More recent evidence implicates peripheral site of actions, such as receptors located on sympathetic nerve terminals, receptors located in vascular tissues or in heart muscle, or a combination of all of the above.

In sedated laboratory animals, dose-response studies indicate that HU-210 appeared to be more potent in causing hypotension than in eliciting bradycardia. The maximal decrease in Mean Arterial Blood Pressure (MABP) and in Heart Rate (HR) caused by HU-210 exceeded those of Δ⁹-THC, in correlation with the finding that HU-210 is also more psychoactive than Δ⁹-THC and binds the CB1 receptor with higher affinity.

Additional pharmacological effects of HU-210 were recently reviewed (Ottani A. et al., CNS Drug Rev. 7(2): 131-45, 2001). In general HU-210 is several fold more potent than Δ⁹-THC, in reducing psychomotor function, interfering with cognitive functions, inducing endocrine alterations, interfering or suppressing immune function, altering neurochemical development, and impairing emotional response due to anxiogenic activity. HU-210 has also been found to inhibit sexual behavior, to induce dependence and to have anorexic effects.

HU-211, the full chemical name of which is 1,1-dimethylheptyl-(3S,4S)-7- hydroxy-Δ⁶-tetrahydrocannabinol, was disclosed in U.S. Pat. No. 4,876,276 and subsequently assigned the trivial chemical name dexanabinol (CAS number: 112-924-45-5). At first, potential therapeutic applications of dexanabinol included known attributes of marijuana itself such as anti-emesis, analgesia, and anti-glaucoma, as disclosed in U.S. Pat. No. 4,876,276.

Further research revealed unexpected properties for dexanabinol and its derivatives, especially neuroprotective properties. It was later established that novel synthetic compounds could block the NMDA receptor, as disclosed in U.S. Pat. Nos. 5,284,867, 5,521,215 and 6,096,740. The capacity of dexanabinol and some of its analogues to block glutamate neurotoxicity has therapeutic implications for treating acute injuries to the central nervous system, including mechanical trauma, prolonged seizures, deprivation of glucose supply, and compromised blood supply (e.g. cardiac arrest or stroke), as well as chronic degenerative disorders characterized by neuronal loss (e.g. Alzheimer's disease, Huntington's chorea, and Parkinson's disease), and poisoning affecting the central nervous system (e.g. strychnine, picrotoxin and organophosphorous poisoning).

Dexanabinol and its analogues appear to share anti-oxidative, immunomodulatory and anti-inflammatory properties in addition to their capacity to block the NMDA receptor, as disclosed in U.S. Pat. Nos. 5,932,610, 6,331,560 and 6,545,041. The convergence of such diverse and crucial therapeutic activities in the dexanabinol molecule made it an excellent candidate for prevention or treatment of a variety of clinical conditions. Currently, the neuroprotective effects of dexanabinol are being assessed in clinical trials. One trial is being conducted to determine the efficacy of dexanabinol in patients suffering from traumatic brain injuries (TBI), while in another trial dexanabinol is administered during surgical procedures to assess its preventive or ameliorative effect on post-operative cognitive impairment.

U.S. Pat. No. 5,284,867 to Kloog et al. discloses HU-211, essentially free of the (3R, 4R) enantiomer. The laboratory scale synthesis of HU-211 is disclosed in U.S. Pat. No. 4,876,276 to Mechoulam et al., wherein the dexanabinol compound is claimed. Kloog does not teach the synthesis of HU-211, but discloses in its Preparatory Example that the acetylation of HU-211 results in a mixture of non-acetylated (HU-211), mono-acetylated (HU-247 and compound B) and bi-acetylated (compound B) derivatives of dexanabinol, and teaches that the acetylated compounds A and B can be reduced to recover the starting material HU-211.

A laboratory scale batch of HU-211 was prepared according to a slightly improved version of Mechoulam's original synthetic procedure. This batch contains 91.1% HU-211 and 0.26% HU-210, which results in 99.4% enantiomeric excess. This sample is either equivalent to or superior to the sample prepared by Kloog.

The prepared sample was further tested for suppression of spontaneous activity, hypothermia, and catalepsy measured in the mice tetrad assay, and clearly produced adverse effects. It caused a drastic drop in rectal temperature of about 6° C., and almost totally inhibited spontaneous locomotion, with the animals crossing only about 6% of the original number of squares they would normally traverse over the same period of time. Finally, it caused significant catalepsy, with the animals remaining immobile on the beam for almost 30 seconds.

It was previously disclosed that the compound HU-211 could be produced on the laboratory scale in reported enantiomeric excess (e.e.) of at least 99.8% over HU-210 (Mechoulam R. et al., Tetrahedron Asymmetry 1(5): 315-8, 1990). The synthetic and analytical methods that were used to generate those data were not sufficiently reliable to ensure that such a high enantiomeric excess could reproducibly be attained.

As already stated, two parameters will determine the stereospecificity of the final synthetic cannabinoid prepared according to scheme 1. First, the chirality of the starting material and second its enantiomeric purity. Thus, it is expected that using (+)-α-pinene of 95% enantiomeric excess will lead to synthesis of a (3S,4S) THC type compound with the same level of enantiomeric purity. However, the synthetic route for the preparation of THC-type compounds allows for stereochemical purification through recrystallization at two steps, for the 4-oxo-myrtenyl-pivalate and for the final compound. This observation made possible the synthesis on a laboratory scale of the enantiomers in e.e. of 99.8%, as determined by HPLC analysis. Small-scale preparation of HU-211 opened the way to the study of its properties in numerous in vitro and in vivo systems. This research led to the discovery of HU-211 multifaceted therapeutic characteristics which have been above described.

The quantitative criterion of the minimum acceptable degree of optical purity of an intended therapeutic enantiomer is dictated by the pharmacological potency of the contamination. The higher the psychotropic activity of the enantiomer, the stricter the requirement for optical purity. The enantiomeric pair HU-210 and HU-211 is an extreme case in point and the highly potent psychotropic effects of HU-210 require that HU-211 should be of very high enantiomeric purity. During clinical trials, therapeutic dosages for humans have been shown to range from tens to hundreds of milligrams per subject, requiring that for pharmaceutical use HU-211 must actually be of enantiomeric purity even higher than any reported previously. Furthermore, for pharmaceutical use reproducibility of the synthetic procedures, adherence to product specifications and the ability to produce the compound on a large scale are necessary features of the active pharmaceutical ingredient. There remains a recognized need for a commercially reproducible dexanabinol compound of high enantiomeric purity for clinical uses.

### SUMMARY OF THE INVENTION

The present invention now provides enantiomerically pure dexanabinol for use as an active ingredient in pharmaceutical compositions for clinical applications.

The present invention encompasses a compound of formula (I):

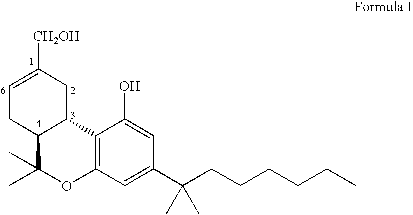

having the (3S,4S) configuration and being in enantiomeric excess of at least 99.90% over the (3R,4R) enantiomer, or a pharmaceutically acceptable salt, ester or solvate of this compound. Preferably, this compound or its pharmaceutically acceptable salt, ester or solvate, is in enantiomeric excess of at least 99.92% over the (3R,4R) enantiomer. More preferably, the compound of formula (I) or its pharmaceutically acceptable salt, ester or solvate, is in enantiomeric excess of at least 99.95% over the (3R,4R) enantiomer. Most preferably, the compound of formula (I) or its pharmaceutically acceptable salt, ester or solvate, is in enantiomeric excess of at least 99.97% over the (3R,4R) enantiomer.

The present invention provides a compound of formula (I) as above defined, wherein the absolute enantiomeric amount of the (3S,4S) enantiomer, or a pharmaceutically acceptable salt, ester or solvate thereof, is at least 99.95% and the (3R,4R) enantiomer is 0.05% or less. Preferably, the compound of formula (I) or its pharmaceutically acceptable salt, ester or solvate, is present in absolute enantiomeric amount of at least 99.96% whereas the (3R,4R) enantiomer is 0.04% or less. More preferably, the compound of formula (I) or its pharmaceutically acceptable salt, ester or solvate, is present in absolute enantiomeric amount of at least 99.97% whereas the (3R,4R) enantiomer is 0.03% or less. Most preferably, the compound of formula (I) or its pharmaceutically acceptable salt, ester or solvate, is present in absolute enantiomeric amount of at least 99.98% whereas the (3R,4R) enantiomer is 0.02% or less.

The present invention further encompasses pharmaceutical compositions comprising as an active ingredient dexanabinol, a compound of formula (I):

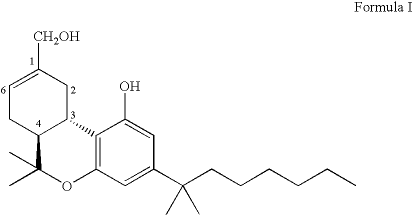

having the (3S,4S) configuration and being in enantiomeric excess of at least 99.90% over the (3R,4R) enantiomer, or a pharmaceutically acceptable salt, ester or solvate of said compound. Preferably, this active ingredient is in enantiomeric excess of at least 99.92% over the (3R,4R) enantiomer. More preferably, this active ingredient is in enantiomeric excess of at least 99.95% over the (3R,4R) enantiomer. Most preferably, this active ingredient is in enantiomeric excess of at least 99.97% over the (3R,4R) enantiomer.

The present invention further encompasses pharmaceutical compositions comprising as an active ingredient dexanabinol, a compound of formula (I) as above defined, wherein the absolute enantiomeric amount of the (3S,4S) enantiomer, or a pharmaceutically acceptable salt, ester or solvate of this compound, is at least 99.95% and the (3R,4R) enantiomer is 0.05% or less. Preferably, the compound of formula (I) or its pharmaceutically acceptable salt, ester or solvate, is present in absolute enantiomeric amount of at least 99.96% whereas the (3R,4R) enantiomer is 0.04% or less. More preferably, the compound of formula (I) or its pharmaceutically acceptable salt, ester or solvate, is present in absolute enantiomeric amount of at least 99.97% whereas the (3R,4R) enantiomer is 0.03% or less. Most preferably, the compound of formula (I) or its pharmaceutically acceptable salt, ester or solvate, is present in absolute enantiomeric amount of at least 99.98% whereas the (3R,4R) enantiomer is 0.02% or less.

The present invention also relates to pharmaceutical compositions comprising as an active ingredient enantiomerically pure dexanabinol, having the (3S,4S) configuration and being in enantiomeric excess of at least 99.90% over the (3R,4R) enantiomer, or a pharmaceutically acceptable salt, ester or solvate of the compound as above defined, and further comprising a pharmaceutically acceptable diluent, carrier or excipient necessary to produce a physiologically acceptable and stable formulation. Preferably, the enantiomerically pure dexanabinol, or its pharmaceutically acceptable salt, ester or solvate, is in enantiomeric excess of at least 99.92% over the (3R,4R) enantiomer. More preferably, the enantiomerically pure dexanabinol, or its pharmaceutically acceptable salt, ester or solvate, is in enantiomeric excess of at least 99.95% over the (3R,4R) enantiomer. Most preferably, the enantiomerically pure dexanabinol, or its pharmaceutically acceptable salt, ester or solvate, is in enantiomeric excess of at least 99.97% over the (3R,4R).

The present invention also relates to pharmaceutical compositions comprising as an active ingredient enantiomerically pure dexanabinol, or a pharmaceutically acceptable salt, ester or solvate of the compound as above defined, having the (3S,4S) configuration and being present in absolute enantiomeric amount of at least 99.95%, and further comprising a pharmaceutically acceptable diluent, carrier or excipient necessary to produce a physiologically acceptable and stable formulation. Preferably, the enantiomerically pure dexanabinol, or its pharmaceutically acceptable salt, ester or solvate, is present in absolute enantiomeric amount of at least 99.96% whereas the (3R,4R) enantiomer is 0.04% or less. More preferably, the enantiomerically pure dexanabinol, or its pharmaceutically acceptable salt, ester or solvate, is present in absolute enantiomeric amount of at least 99.97% whereas the (3R,4R) enantiomer is 0.03% or less. Most preferably, the enantiomerically pure dexanabinol, or its pharmaceutically acceptable salt, ester or solvate, is present in absolute enantiomeric amount of at least 99.98% whereas the (3R,4R) enantiomer is 0.02% or less.

The pharmaceutical compositions can be administered by any conventional and appropriate route including oral, parenteral, intravenous, intramuscular, subcutaneous, transdermal, intrathecal, rectal or intranasal.

Prior to their use as medicaments for preventing, alleviating or treating an individual in need thereof, the pharmaceutical compositions may be formulated in unit dosage form. The selected dosage of active ingredient depends upon the desired therapeutic effect, the route of administration and the duration of treatment desired.

A further embodiment of the present invention provides a method of preventing, alleviating or treating a patient for indications including but not limited to acute neurological disorders, chronic degenerative diseases, CNS poisoning, cognitive impairment, inflammatory diseases or disorders, autoimmune diseases or disorders, pain, emesis, glaucoma and wasting syndromes, by administering to said patient a prophylactically and/or therapeutically effective amount of one of the enantiomerically pure dexanabinol compounds described herein or a pharmaceutical composition that contains such compounds as above defined wherein enantiomerically pure dexanabinol is in enantiomeric excess of at least 99.90% over the (3R,4R) enantiomer. Preferably, the enantiomerically pure dexanabinol, or its pharmaceutically acceptable salt, ester or solvate, is in enantiomeric excess of at least 99.92% over the (3R,4R) enantiomer. More preferably, the enantiomerically pure dexanabinol, or its pharmaceutically acceptable salt, ester or solvate, is in enantiomeric excess of at least 99.95% over the (3R,4R) enantiomer. Most preferably, the enantiomerically pure dexanabinol, or its pharmaceutically acceptable salt, ester or solvate, is in enantiomeric excess of at least 99.97% over the (3R,4R) enantiomer.

A further embodiment of the present invention provides a method of preventing, alleviating or treating a patient for indications including but not limited to acute neurological disorders, chronic degenerative diseases, CNS poisoning, cognitive impairment, inflammatory diseases or disorders, autoimmune diseases or disorders, pain, emesis, glaucoma and wasting syndromes, by administering to said patient a prophylactically and/or therapeutically effective amount of one of the enantiomerically pure dexanabinol compounds described herein or a pharmaceutical composition that contains such compounds as above defined wherein enantiomerically pure dexanabinol is present in absolute enantiomeric amount of at least 99.95% whereas the (3R,4R) enantiomer is 0.05% or less. Preferably, the enantiomerically pure dexanabinol, or its pharmaceutically acceptable salt, ester or solvate, is present in absolute enantiomeric amount of at least 99.96% whereas the (3R,4R) enantiomer is 0.04% or less. More preferably, the enantiomerically pure dexanabinol, or its pharmaceutically acceptable salt, ester or solvate, is present in absolute enantiomeric amount of at least 99.97% whereas the (3R,4R) enantiomer is 0.03% or less. Most preferably, the enantiomerically pure dexanabinol, or its pharmaceutically acceptable salt, ester or solvate, is present in absolute enantiomeric amount of at least 99.98% whereas the (3R,4R) is 0.02% or less.

A further embodiment of the present invention provides use for the manufacture of a medicament for preventing, alleviating or treating acute neurological disorders, chronic degenerative diseases, CNS poisoning, cognitive impairment, inflammatory diseases or disorders, autoimmune diseases or disorders, pain, emesis, glaucoma and wasting syndromes, of one of the enantiomerically pure dexanabinol compounds described herein wherein enantiomerically pure dexanabinol is in enantiomeric excess of at least 99.90% over the (3R,4R) enantiomer. Preferably, the enantiomerically pure dexanabinol, or its pharmaceutically acceptable salt, ester or solvate, is in enantiomeric excess of at least 99.92% over the (3R,4R) enantiomer. More preferably, the enantiomerically pure dexanabinol, or its pharmaceutically acceptable salt, ester or solvate, is in enantiomeric excess of at least 99.95% over the (3R,4R) enantiomer. Most preferably, the enantiomerically pure dexanabinol, or its pharmaceutically acceptable salt, ester or solvate, is in enantiomeric excess of at least 99.97% over the (3R,4R) enantiomer.

A further embodiment of the present invention provides use for the manufacture of a medicament for preventing, alleviating or treating acute neurological disorders, chronic degenerative diseases, CNS poisoning, cognitive impairment, inflammatory diseases or disorders, autoimmune diseases or disorders, pain, emesis, glaucoma and wasting syndromes, of one of the enantiomerically pure dexanabinol compounds described herein wherein enantiomerically pure dexanabinol is present in absolute enantiomeric amount of at least 99.95% whereas the (3R,4R) enantiomer is 0.05% or less. Preferably, the enantiomerically pure dexanabinol, or its pharmaceutically acceptable salt, ester or solvate, is present in absolute enantiomeric amount of at least 99.96% whereas the (3R,4R) enantiomer is 0.04% or less. More preferably, the enantiomerically pure dexanabinol, or its pharmaceutically acceptable salt, ester or solvate, is present in absolute enantiomeric amount of at least 99.97% whereas the (3R,4R) enantiomer is 0.03% or less. Most preferably, the enantiomerically pure dexanabinol, or its pharmaceutically acceptable salt, ester or solvate, is present in absolute enantiomeric amount of at least 99.98% whereas the (3R,4R) is 0.02% or less.

These and additional benefits and features of the invention could be better understood by those skilled in the art with reference to the following detailed description taken in conjunction with the figures and non-limiting examples.

### BRIEF DESCRIPTION OF THE FIGURES

The accompanying drawings, which are incorporated in and form a part of the specification, illustrate certain embodiments of the present invention, and together with the description serve to explain the principles of the invention. In the drawings:

FIG. 1 shows expanded HPLC chromatograms of four pharmaceutical grade, large scale, batches of enantiomerically pure dexanabinol.

FIG. 2 shows the profile of dexanabinol plasma concentration along time, following single or multiple injections of specified doses in the various species tested.

FIG. 3 shows the profile of dexanabinol concentrations in plasma and brain of rats injected with 4 mg/kg of the drug.

### DETAILED DESCRIPTION OF THE INVENTION

The present invention provides ultrapure dexanabinol characterized by an enantiomeric excess of at least 99.90%, preferably 99.92%, more preferably 99.95% and most preferably 99.97%, for use as an active pharmaceutical ingredient in compositions for clinical applications.

The enantiomerically pure dexanabinol of the present invention is further characterized by an absolute enantiomeric amount of at least 99.95%, preferably 99.96%, more preferably 99.97% and most preferably 99.98%. The respective absolute enantiomeric amount of HU-210 is 0.05% or less, preferably 0.04% or less, more preferably 0.03% or less and most preferably 0.02% or less.

In the present specification and claims which follow the terms HU-211, dexanabinol, 1,1-dimethylheptyl-(3S,4S)-7-hydroxy-Δ⁶-tetrahydrocannabinol and (+)(6aS, 10aS)-6,6-dimethyl-3-(1,1-dimethylheptyl)-1-hydroxy-6a,7,10,10a-tetrahydro-6H-dibenzo[b,d]pyran-9-methanol are alternatively used to represent the same chemical entity.

In the present specification and claims which follow the terms HU-210, 1,1-dimethylheptyl-(3R,4R)-7-hydroxy-Δ⁶-tetrahydrocannabinol and (−)(6aR, 10aR)-6,6-dimethyl-3-(1,1-dimethylheptyl)-1-hydroxy-6a,7,10,10a-tetrahydro-6H-dibenzo[b,d]pyran-9-methanol are alternatively used to represent the same chemical entity.

In the present specification the terms “enantiomerically pure”, “enantiomeric purity” and “optical purity” are used alternatively to reflect the fact that one enantiomer, generally (3S,4S) when referring to compound of formula (I), is found in the composition in greater proportion in relation to its mirror image. The proportion between two enantiomers can be expressed either by the enantiomeric excess or by the absolute proportion of each enantiomer.

In the present specification and claims which follow the term “enantiomeric excess” (e.e.) represents the percent excess of one enantiomer over the other and is calculated using the following equation:
Percent e.e.=100*([enantiomer 1]−[enantiomer 2])/([enantiomer 1]+[enantiomer 2]).

Thus the formula used to calculate the enantiomeric excess of dexanabinol over HU-210 is 100*([HU-211]−[HU-210])/([HU-211]+[HU-210]), wherein the concentration of the enantiomers is determined by HPLC and expressed as percent weight by weight.

In the present specification and claims which follow the term “absolute enantiomeric amount” represents the percent of each enantiomer and is calculated using the following equation:

Absolute enantiomeric amount=100*[enantiomer 1]/([enantiomer 1]+[enantiomer 2]), wherein the concentration of the enantiomers is determined by HPLC and expressed as percent weight by weight.

Psychoactive cannabinoids have a variety of pharmacological effects that are evoked through interactions with the CB1 receptors predominantly expressed in the brain. The central nervous system effects that occur after in vivo exposure to HU-210 include euphoria, hypokinesia, hypothermia, anti-nociception, and catalepsy. Hypokinesia, hypothermia, anti-nociception, and catalepsy are the activities that form the basis of the “Tetrad assay” of CB1 mediated activity. HU-210 was shown to be a hundred times more psychoactive than Δ⁹-THC, the major natural component of hashish, and a thousand times more psychoactive than HU-211 in a series of animal tests (Mechoulam R. et al., Tetrahedron Asymmetry 1(5): 315-8, 1990). The threshold for psychomimetic effects of oral THC in humans is in the range of 10-15 mg THC in an adult, i.e from about 0.16 to about 0.25 mg THC/kg body weight for adults of approximately 60 kg (“Cannabis and Cannabinoids, pharmacology, toxicology and therapeutic potential” edited by Grotenhermen and Russo, the Haworth Integrative Healing Press; Chapter 31 pp 345 and 351). Since HU-210 psychoactivity in animals is approximately 200 fold higher than Δ⁹-THC, the maximal tolerated dose of HU-210 theoretically should be less than 1.25 μg/kg body weight.

In other words, for a dose of a medicament to be devoid of psychoactivity, it should contain less than 1.25 μg/kg body weight of HU-210, anda dose of 150 mg dexanabinol should contain less than about 0.05% of HU-210 w/w. Dexanabinol must be in enantiomeric excess of 99.9% or more in order to be within the safety margin as far as HU-210 side effects are concerned.

Dexanabinol according to the present invention was tested for suppression of spontaneous activity, hypothermia, and catalepsy in the mice tetrad assay. Unlike the Kloog sample, this sample had no adverse effect in mice at 50 mg/kg intravenously. This demonstrates the superiority of the present dexanabinol over the compound disclosed in Kloog.

As previously noted, effects due to CB1 activation are not limited to psychomimetic effects, but cardiac manifestations also are elicited. The effects of the stereochemically pure HU-210 on blood pressure and heart rate were determined in rats by Vidrio et al. (Vidrio, H. et al., J Cardiovasc Pharmacol, 28(2): 332-6, 1996). In pentobarbital-anesthetized animals, the compound produced dose-related, long-lasting hypotension and bradycardia at doses between 10 and 1,000 μg/kg. Blood pressure began to decrease immediately after drug administration, and neither previous vagotomy or pretreatment with 6-hydroxydopamine had any effect on the hypotensive effects of the drug. HU-210 also decreased blood pressure and heart rate in conscious rats. The effects of drug administration were prolonged; the hypotensive effects lasted for 2 hours, whereas bradycardia was still present 8 hours after drug administration.

These cardiovascular effects were further studied several years later by Wagner et al. (Wagner J. A. et al., Eur. J. Pharmacol. 423(2-3): 203-10, 2001). In studies performed in rats, HU-210 at doses of 10 μg/kg i.v. reduced mean blood pressure by 57±5 mm Hg by decreasing cardiac index from 37±1 to 23±1 mL/min/100 g (p<0.05) without significantly affecting systemic vascular resistance index. In these studies, HU-210 elicited a similar decrease in blood pressure following ganglionic blockade and vasopressin infusion. More recently, investigators from the University of Edinburgh have reported not only cardiovascular effects, but direct cerebrovascular effects of HU-210 in a rat model (Simpson A. M. et al., J Cerebral Blood Flow and Metabolism 25: S581, 2005). In these studies, not only did HU-210 significantly decrease the mean arterial blood pressure (from 142±1 to 105±6 mm Hg) and heart rate (from 484±6 to 232±18 beats/min) but the PaCO₂ also significantly increased (from 32±0.7 to 45.7±1.6 mmHg). These effects were established at 5 minutes post-infusion, and persisted relatively unchanged through the experimental period. HU-210 induced significant decreases in cerebral glucose use in all brain regions examined (hippocampus, striatum and three neocortical brain areas), ranging from −19% in frontal cortex (from 69±1 to 55±3 μmol.100 g−1.min-1) to −31% in occipital cortex (from 60±1 to 41±2 μmol.100 g−1.min−1). Additional studies performed by Lake et al. have confirmed that dexanabinol is devoid of cardiovascular effects (Lake K. D. et al, JPET 281: 1030-7, 1997). The hypotensive effect of HU-210, however, is detected at an administered dose as low as 0.001 mg/kg i.v. HU-210.

The enantiomeric purity of the active ingredient is determined by types of tests known in the art, for example chiral HPLC methods and reverse phase HPLC. The present invention required development of novel modified chiral HPLC methods (adapted from Levin S. et al., Journal of Chromatography A. 654: 53-64, 1993) exemplified hereinbelow, in conjunction with RP-HPLC. Analytical methods previously disclosed in the art were not validatable and did not provide reliable and reproducible results. Among the reasons for the inaccuracy of previously known methods is their failure to resolve certain impurities that can elute with parameters overlapping those of the desired product itself.

The scaled up synthetic procedures according to the present invention, generally adhere to the synthetic schemes used previously, with modifications to enable good manufacturing practice. The improvements implemented were required to obtain pharmaceutical grade dexanabinol reproducibly and with the required elevated standard of enantiomeric purity.

The improvements introduced in the synthetic procedures according to the present invention over the previously known laboratory scale procedure, as described in U.S. Pat. No. 4,876,276, are evident to persons skilled in the art and include scale-up ability, improved yield, simplified process, reduced use of toxic chemicals or dangerous reagents all leading to a safer and more cost effective production.

Moreover, it is now disclosed that the crystallization performed at the final step of the synthesis is crucial for the purity of dexanabinol. Previously disclosed procedures for the synthesis of dexanabinol (U.S. Pat. No. 4,876,276) did not teach or suggest the importance of the final crystallization step in achieving the enantiomeric purity required for pharmaceutical or clinical grade material. On the contrary, emphasis was drawn to crystallization of 4-oxomyrtenyl pivalate (compound 4 in scheme 3) as pivotal for the enantiomeric purity of the final compound. Furthermore, it is now disclosed that the selection of solvent or mother liquor for the final crystallization may affect the purity of the product, as well as the efficiency of the crystallization.

The synthetic process for the preparation of dexanabinol combines two approaches for obtaining the desired enantiomer; first the utilization of enantiomerically enriched starting material, namely (+)-α-pinene, in a stereoselective multistep synthesis and then the separation of the partially resolved racemic mixture into their enantiomeric constituent using crystallization. The use of commercially available high purity (+)-α-pinene can ensure enantiomeric excess of about 98% for the final synthetic product dexanabinol, a level not high enough however if HU-210 can constitute as much as 1% of the final mixture. Moreover, it should be noted that such pure starting material is very expensive and therefore economically suitable for laboratory scale synthesis only. Economic constraints of industrial scale synthesis necessitate the use of (+)-α-pinene of lower enantiomeric purity of about 90%, further increasing the importance of the resolution by crystallization performed in the final stages of synthesis to reduce or eliminate the presence of HU-210 in the final product.

Though crystallization methods are widely employed for the separation and purification of enantiomers, and general guidelines have been established (Collet A., Enantiomer 4: 157-72, 1999), the determination of the optimal conditions still remains unpredictable and mainly empirical. The discovery, as exemplified herein below, that acetonitrile is superior to previously published solvents derived from the need to further purify the first commercial batch of dexanabinol prepared according to previously published procedures. This observation suggests that previously published solvents are appropriate for enantiomeric separation and purification in synthesis performed only on laboratory scale and/or using higher purity grade starting materials that are economically affordable on laboratory scale only.

As is known in the art, the conditions to achieve enantiomeric separation depend whether the object of the process is to maximize recovery and/or purity and they include parameters such as the composition of the solvent, the concentration, and the temperature. These parameters can be determined by one skilled in the art of recrystallization using the particular compound of which separation of the enantiomers is desired. Such additional solvents or mixtures of solvents for purifying the (3S,4S) enantiomer are embraced in the invention herein.

The purity can be increased, if necessary, by repeating the final crystallization step with acetonitrile. Additional round(s) of recrystallization is a standard procedure and expected necessity if the initial purity is not adequate and does not fall within the specifications defined by the intended use of the product. Such additional means for purifying the (3S,4S) enantiomer are embraced in the invention herein.

Dexanabinol is capable of further forming pharmaceutically acceptable salts and esters. “Pharmaceutically acceptable salts and esters” means any salt and ester that is pharmaceutically acceptable and has the desired pharmacological properties. Such salts include salts that may be derived from an inorganic or organic acid, or an inorganic or organic base, including amino acids, which is not toxic or otherwise unacceptable. The present invention also includes within its scope solvates of dexanabinol and salts thereof, for example, hydrates. In the present specification the term “prodrug” represents compounds which are rapidly transformed in vivo to dexanabinol, for example by hydrolysis in the blood. All of these pharmaceutical forms are intended to be included within the scope of the present invention.

Water-soluble derivatives of dexanabinol were synthesized and investigated over the years. They can be used as prodrugs, or active analogs depending on their hydrolytic and enzymatic stability and on their intrinsic activity. The two hydroxyl groups present in the dexanabinol molecule were targeted for modifications and various polar combinations or combinations bearing a permanent charge were synthesized as esters at the allylic or phenolic hydroxyls. Modifications included glycinate and N-substituted glycinates, esters of amino acids containing tertiary or quaternary heterocyclic nitrogen, phosphates, and hemiesters of dicarboxylic acids. Synthetic procedures, water solubility and stability in buffers and human plasma, as well as in vivo tissue distribution of water-soluble dexanabinol analogues, were abundantly described (U.S. Pat. No. 6,096,740; Pop E. et al., Pharm. Res. 13: 62-9, 1996; Pop E. et al., Pharm. Res. 13: 469-75, 1996; Pop E. et al., J. Pharm. Sci. 88: 1156-60, 1999; Pop E. et al., Pharmazie 55: 167-71, 2000). Several derivatives possess the required properties to be used as water-soluble prodrugs: they are soluble and fairly stable in water, but rapidly hydrolyze in human blood into parent dexanabinol.

In the present specification and claims which follow “prophylactically effective” is intended to qualify the amount of compound which will achieve the goal of prevention, reduction or eradication of the risk of occurrence of the disorder, while avoiding adverse side effects. The term “therapeutically effective” is intended to qualify the amount of compound that will achieve, with no adverse effects, alleviation, diminished progression or treatment of the disorder, once the disorder cannot be further delayed and the patients are no longer asyinptomatic. The compositions of the present invention are prophylactic as well as therapeutic.

The “individual” or “patient” for purposes of treatment includes any human or mammalian subject affected by any of the diseases where the treatment has beneficial therapeutic impact.

By virtue of the anti-inflammatory and immunomodulatory properties of dexanabinol, it will be recognized that the compositions according to the present invention will be useful for treating indications having an inflammatory or autoimmune mechanism involved in their etiology or pathogenesis. Such diseases or disorders are exemplified by multiple sclerosis, amyotrophic lateral sclerosis, systemic lupus erythematosis, myasthenia gravis, diabetes mellitus type I, sarcoidosis; skeletal and connective tissue disorders including arthritis, rheumatoid arthritis, osteoarthritis and rheumatoid diseases; ocular inflammation related disorders; skin related disorders including psoriasis, pemphigus and related syndromes, delayed-type hypersensitivity and contact dermatitis; respiratory diseases including cystic fibrosis, chronic bronchitis, emphysema, chronic obstructive pulmonary disease, asthma, allergic rhinitis or lung inflammation, idiopathic lung fibrosis, tuberculosis, and alveolitis; kidney diseases including renal ischemia, nephrites, nephritic syndromes and nephrosis characterized by glomerular nephritides; liver diseases both acute and chronic such as cirrhosis; gastrointestinal diseases including inflammatory bowel diseases, ulcerative colitis, Crohn's disease and gastritis, polyposis and cancer of the bowel, especially the colon; infectious diseases generated by certain bacterial, viral and parasitic invasion and sepsis that might result from injury; and post-operative complications following angioplasty, circulatory recovery techniques, prosthetic implants and tissue or organ transplants, including graft rejection.

By virtue of the neuroprotective properties of dexanabinol, it will be recognized that the compositions according to the present invention will be useful in treating acute neurological disorders, resulting either from ischemic or traumatic damage, including but not limited to stroke, head trauma and spinal cord injury. The composition of the present invention may also be effective in preventing or treating certain chronic degenerative diseases that are characterized by gradual selective neuronal loss such as Parkinson's disease, Alzheimer's disease, AIDS dementia, Huntington's chorea, and prion-associated neurodegeneration. The compositions may further be effective in prevention or diminution of cognitive impairment for instance post-operative, disease induced, virally induced, therapy induced or neonatal cognitive impairment and of CNS poisoning, for instance by strychnine, picrotoxin or organophosphorous compounds.

By virtue of the analgesic properties of dexanabinol, it will be recognized that the compositions according to the present invention will be useful in treating pain including peripheral, neuropathic and referred pain.

The compositions of the present invention will also be effective in relieving emesis and treating glaucoma, retinal eye diseases and cachexia due to acquired immunodeficiency syndrome, neoplasia or other wasting diseases.

The present invention provides a compound of formula (I): 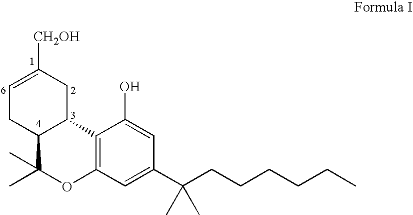
having the (3S,4S) configuration and being in enantiomeric excess of at least 99.90% over the (3R,4R) enantiomer, or a pharmaceutically acceptable salt, ester or solvate of said compound.

Preferably the compound of formula (I), dexanabinol, or a pharmaceutically acceptable salt, ester or solvate of said compound, is in enantiomeric excess of at least 99.92% over the (3R,4R) enantiomer.

More preferably the compound of formula (I), dexanabinol, or a pharmaceutically acceptable salt, ester or solvate of said compound, is in enantiomeric excess of at least 99.95% over the (3R,4R) enantiomer.

Most preferably the compound of formula (I), dexanabinol, or a pharmaceutically acceptable salt, ester or solvate of said compound, is in enantiomeric excess of at least 99.97% over the (3R,4R) enantiomer.

The present invention provides a compound of formula (I) as above defined, wherein the absolute enantiomeric amount of the (3S,4S) enantiomer is at least 99.95% and the (3R,4R) enantiomer is 0.05% or less.

Preferably the compound of formula (I), dexanabinol, or a pharmaceutically acceptable salt, ester or solvate of said compound, is present in absolute enantiomeric amount of at least 99.96% and the (3R,4R) enantiomer is 0.04% or less.

More preferably the compound of formula (1), dexanabinol, or a pharmaceutically acceptable salt, ester or solvate of said compound, is present in absolute enantiomeric amount of at least 99.97% and the (3R,4R) enantiomer is 0.03% or less.

Most preferably the compound of formula (I), dexanabinol, or a pharmaceutically acceptable salt, ester or solvate of said compound, is present in absolute enantiomeric amount of at least 99.98% and the (3R,4R) enantiomer is 0.02% or less.

The present invention provides pharmaceutical compositions comprising as an active ingredient dexanabinol, a compound of formula (I): 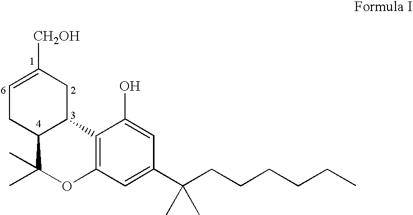
having the (3S,4S) configuration and being in enantiomeric excess of at least 99.90% over the (3R,4R) enantiomer, or a pharmaceutically acceptable salt, ester or solvate of said compound.

Preferably the active ingredient of the above-defined pharmaceutical composition, dexanabinol, or a pharmaceutically acceptable salt, ester or solvate of said compound, is in enantiomeric excess of at least 99.92% over the (3R,4R) enantiomer.

More preferably the active ingredient of the above-defined pharmaceutical composition, dexanabinol, or a pharmaceutically acceptable salt, ester or solvate of said compound, is in enantiomeric excess of at least 99.95% over the (3R,4R) enantiomer.

Most preferably the active ingredient of the above-defined pharmaceutical composition, dexanabinol, or a pharmaceutically acceptable salt, ester or solvate of said compound, is in enantiomeric excess of at least 99.97% over the (3R,4R) enantiomer.

The present invention provides pharmaceutical compositions comprising as an active ingredient a compound of formula (I) as above defined, wherein the absolute enantiomeric amount of the (3S,4S) enantiomer is at least 99.95% and the (3R,4R) enantiomer is 0.05% or less.

Preferably the active ingredient of the above-defined pharmaceutical composition is present in absolute enantiomeric amount of at least 99.96% and the (3R,4R) enantiomer is 0.04% or less.

More preferably the active ingredient of the above-defined pharmaceutical composition is present in absolute enantiomeric amount of at least 99.97% and the (3R,4R) enantiomer is 0.03% or less.

Most preferably the active ingredient of the above-defined pharmaceutical composition is present in absolute enantiomeric amount of at least 99.98% and the (3R,4R) enantiomer is 0.02% or less.

The present invention also provides pharmaceutical compositions comprising as an active ingredient an enantiomerically pure compound of formula (I) having the (3S,4S) configuration and being in enantiomeric excess of at least 99.90%, preferably 99.92%, more preferably 99.95% and most preferably 99.97%, over the (3R,4R) enantiomer, further comprising a pharmaceutically acceptable diluent or carrier.

The present invention also provides pharmaceutical compositions comprising as an active ingredient an enantiomerically pure compound of formula (I) having the (3S,4S) configuration and being present in absolute enantiomeric amount of at least 99.95%, preferably 99.96%, more preferably 99.97% and most preferably 99.98%, further comprising a pharmaceutically acceptable diluent or carrier.

The pharmaceutical compositions contain in addition to the active ingredient conventional pharmaceutically acceptable carriers, diluents and excipients necessary to produce a physiologically acceptable and stable formulation. Some compounds of the present invention are characteristically hydrophobic and practically insoluble in water with high lipophilicity, as expressed by their high octanol/water partition coefficient expressed as log P values, and formulation strategies to prepare acceptable dosage forms will be applied. Enabling therapeutically effective and convenient administration of the compounds of the present invention is an integral part of this invention.

For water-soluble derivatives of dexanabinol standard formulations will be utilized. Solid compositions for oral administration such as tablets, pills, capsules, softgels or the like may be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Other solid compositions can be prepared as suppositories, for rectal administration. Liquid forms may be prepared for oral administration or for injection, the term including but not limited to subcutaneous, transdermal, intravenous, intrathecal, intralesional, adjacent to or into tumors, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention may be formed as aerosols, for intranasal and like administration. Topical phanmaceutical compositions of the present invention may be formulated as solution, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Prior to their use as medicaments, the pharmaceutical compositions will generally be formulated in unit dosage. The active dose for humans is generally in the range of from 0.05 mg to about 50 mg per kg body weight, in a regimen of 1-4 times a day. The preferred range of dosage is from 0.1 mg to about 20 mg per kg body weight. However, it is evident to one skilled in the art that dosages would be determined by the attending physician, according to the disease to be treated, its severity, the method and frequency of administration, the patient's age, weight, gender and medical condition, contraindications and the like. The dosage will generally be lower if the compounds are administered locally rather than systematically, and for prevention or chronic treatment rather than for acute therapy.

A further aspect of the present invention provides a method of preventing, alleviating or treating a patient for indications as above described, by administering to said patient a prophylactically and/or therapeutically effective amount of a pharmaceutical composition comprising as an active ingredient enantiomerically pure dexanabinol, having the (3S,4S) configuration and being in enantiomeric excess of at least 99.90%, preferably 99.92%, more preferably 99.95% and most preferably 99.97%, over the (3R,4R) enantiomer, or a pharmaceutically acceptable salt, ester or solvate of said compound as above defined.

A further aspect of the present invention provides a method of preventing, alleviating or treating a patient for indications as above described, by administering to said patient a prophylactically and/or therapeutically effective amount of a pharmaceutical composition comprising as an active ingredient enantiomerically pure dexanabinol, having the (3S,4S) configuration and being present in absolute enantiomeric amount of at least 99.95%, preferably 99.96%, more preferably 99.97% and most preferably 99.98%, or a pharmaceutically acceptable salt, ester or solvate of said compound as above defined.

A further aspect of the present invention relates to the use for the manufacture of a medicament for preventing, alleviating or treating indications as above described, of enantiomerically pure dexanabinol, having the (3S,4S) configuration and being in enantiomeric excess of at least 99.90%, preferably 99.92%, more preferably 99.95% and most preferably 99.98%, over the (3R,4R) enantiomer, or a pharmaceutically acceptable salt, ester or solvate of said compound as above defined.

A further aspect of the present invention relates to the use for the manufacture of a medicament for preventing, alleviating or treating indications as above described, of enantiomerically pure dexanabinol, having the (3S,4S) configuration and being present in absolute enantiomeric amount of at least 99.95%, preferably 99.96%, more preferably 99.97% and most preferably 99.98%, or a pharmaceutically acceptable salt, ester or solvate of said compound as above defined.

The principles of the present invention will be more fully understood by reference to the following examples, which illustrate preferred embodiments of the invention and are to be construed in a non-limitative manner.

### EXAMPLES

### Example 1

### Preparation of Dexanabinol of High Enantiomeric Purity

Dexanabinol was manufactured on a commercial scale in eleven steps starting from (+)-α-pinene (1 in Scheme 3) and involved coupling of 2 main intermediates (Scheme 5), (+) 4-hydroxymyrtenyl pivalate (5 in Scheme 3) and 5′-(1′,1′-dimethylheptyl)-resorcinol (12 in Scheme 4).

The (+) 4-hydroxymyrtenyl pivalate (5) was synthesized from (+)-α-pinene (1) by a 4-step procedure via (+) myrtenol (2). By oxidation of 1 with t-butylhydroperoxide in the presence of SeO₂ on silica gel a mixture of myrtenol and myrtenal was obtained, further reduced to myrtenol by sodium borohydride. Esterification of the myrtenol with pivaloyl chloride gave (+) myrtenol pivalate (3), which by sodium chromate oxidation led to (+)-4-oxomyrtenyl pivalate (4). Borohydride reduction of (4) led to (5).

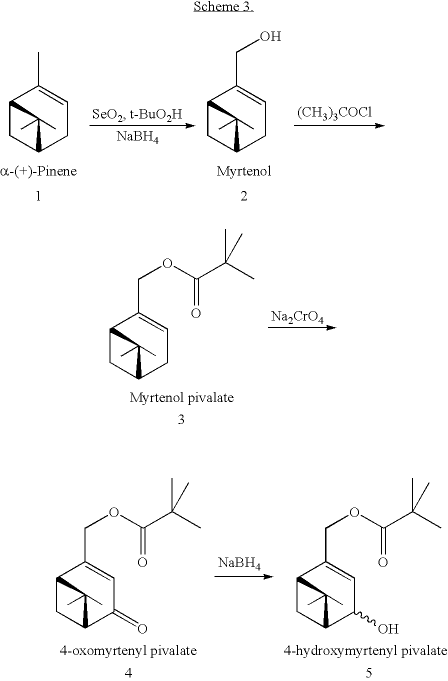

The ⁵-(1′,1′-dimethylheptyl)resorcinol (12) was obtained by a 5-step synthesis which started from 2-octanone (6) and 2,6-dimethoxyphenol (8). In this procedure, (6) was transformed to 2-methyl-2-octanol (7) (Grignard reaction), which then alkylated 8 in methansulfonic acid to give (1′,1′-dimethylheptyl)-2,6-dimethoxyphenol (9). By reacting 9 with diethylphosphonate, the (1′,1′-dimethylheptyl)-2,6-dimethoxyphenyl diethylphosphite (10) was obtained. Treatment of 10 with lithium/ammonia followed by demethylation with boron tribromide of the resulting (1′,1′-dimethylheptyl)-3,5-dimethoxybenzene (11) afforded the 5-substituted resorcinol 12.

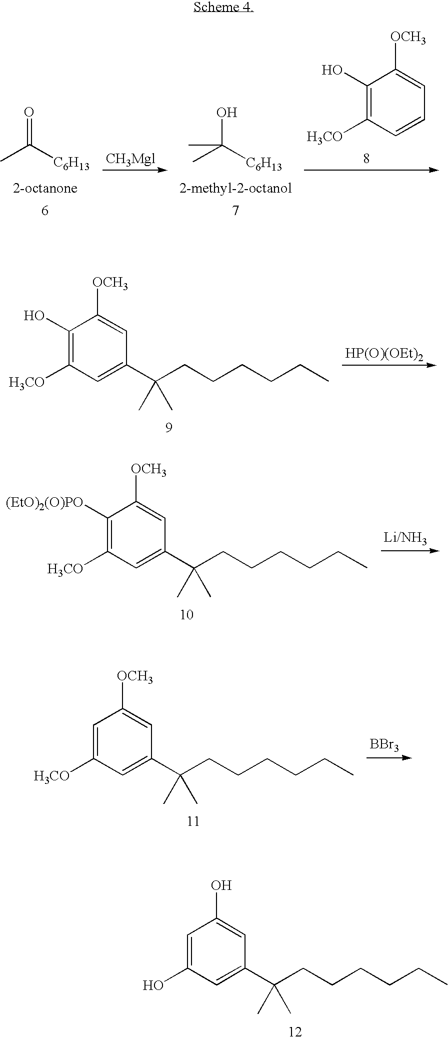

The final steps are described in Scheme 5, wherein the 1,1-dimethyl-heptyl substituent is abbreviated DMH. Coupling of (5) and (12) took place in the presence of boron trifluoride diethyletherate and resulted in the pivaloyl ester of dexanabinol (13), which was subsequently deprotected with lithium aluminium hydride to give the final dexanabinol substance.

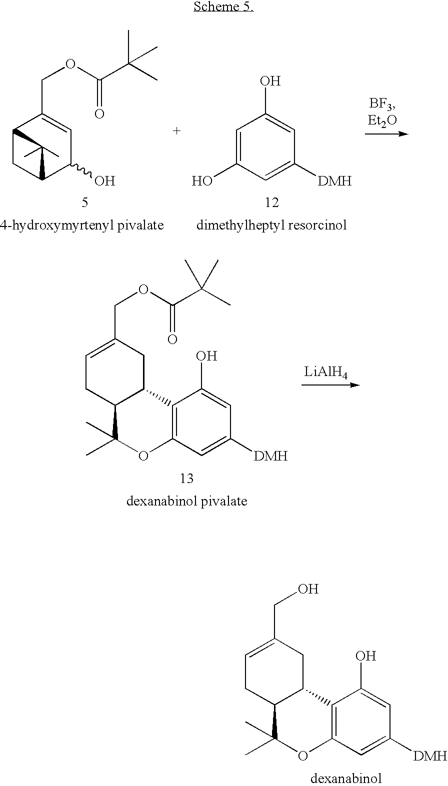

As above stated, the advantages of this process over the previously known laboratory scale procedure, as described in U.S. Pat. No. 4,876,276, are evident to persons skilled in the art and include scale-up ability, improved yield, simplified process, reduced use of toxic chemicals or dangerous reagents all leading to a safer and more cost effective production. The exact conditions of the clinical grade, intermediate scale, synthesis of dexanabinol of high enantiomeric purity are described below.

Step 1 (i): Oxidation of (+)-α-pinene with selenium dioxide

To 0.13 molar equivalents of selenium dioxide on silica gel a 1:3 (w/v) solution of α-pinene (1) in methylene chloride was added. To the mixture were added 0.46 molar equivalents of 70% t-butyl-hydroperoxide at 30° C. The mixture was stirred at 30° C. for 46-50 hours and filtered. The solvents were removed under reduced pressure (50-100 Torr) at 50° C. A mixture of (+) myrtenol and (+) myrtenal resulted.

Step 1 (ii): Reduction of Myrtenal with sodium borohydride

The residue from step 1 was dissolved in methanol, cooled to 0-5° C. and treated with 0.5 molar equivalent of sodium borohydride over 2-3 hours, maintaining the temperature at 0-5° C. The mixture was stirred for an additional 30-60 minutes at 0-5° C., then diluted with one volume of ice water and extracted three times with 0.25 volumes of methylene chloride (each). The combined organic solutions were washed four times with one volume (each) water, dried over 0.05 part anhydrous sodium sulphate, filtered and the solvents were removed under vacuum (50-100 Torr) at 80° C., resulting in (+) myrtenol (2).

Step 2: Esterification of (+) myrtenol with pivaloyl chloride

To a solution of 2 in 1.5 volumes of anhydrous pyridine were added 1.6 molar equivalents of pivaloyl chloride at (−15)-(−10)° C. over 3 hours. The mixture was diluted with 0.2 volumes of pyridine and stirred overnight at 20-25° C. Two volumes of ice water were added to the mixture and the resulting ester was extracted twice with 0.5 volumes (each) of methylene chloride. The solvents (methylene chloride and pyridine) were removed under reduced pressure (50-100 Torr) at 80° C. The resulting myrtenyl pivalate (3) is an oily material. The crude myrtenyl pivalate was used for the following step without further purification, simplifying the process.

Step 3: Oxidation of (+) myrtenyl pivalate with sodium chromate

A solution of 3 in 6 volumes of acetic acid-acetic anhydride (1:1) was treated with 3.3 molar equivalents of sodium chromate at 10-15° C., over 3-5 hours. The mixture was stirred at 20° C. for 16-20 hours, then at 45-50° C. for 24 hours. After cooling at 20-25° C., 0.4 volumes of ice water were added and the mixture was extracted five times with 0.2 volumes (each) of methylene chloride. The combined extracts were washed five times with 0.5 volume (each) of 20% aqueous sodium chloride and concentrated to ⅙ of their volume. The concentrate was washed with one volume of 54% aqueous potassium carbonate and dried over 0.33 parts of anhydrous sodium sulphate. The resulting solution was passed through 0.33 parts of silica gel 60-230 mesh using an eluent of 3.5 volumes of methylene chloride. After removing the solvent under reduced pressure at 50-100 Torr at a final temperature of 80° C., the residue was distilled at 120-165° C. and 0.1-0.15 Torr. The distillate was diluted with two volumes of n-pentane and kept at −20° C. for 40 hours. The resulting crystals of 4-oxomyrtenyl pivalate (4) were filtered, rinsed with cold pentane and dried in a clean, well-ventilated hood. A second crop of material can be obtained from the mother liquors, by removing the solvent and distilling the residue in vacuum and crystallisation from pentane.

Step 4: Reduction of 4-oxomyrtenyl pivalate with sodium borohydride

To a solution of 4 in 16 volumes of methanol were added 1.32 molar equivalents of sodium borohydride at (−15)-(−10)° C. over 2-3 hours. Sodium borohydride advantageously replaces the lithium hydrido-tri-t-butoxylaminate previously used in the laboratory scale process. The molar excess of sodium borohydride versus 4 (1.32) is much reduced as compared to lithium hydrido-tri-t-butoxylaminate (more than 10 fold over 4). The mixture was stirred for another 3 hours at −10° C. and 0.1 volume ice-water was added. Three volumes of water were added and the mixture was extracted with 2.5 volumes of hexane. The extracts were washed three times with 0.4 volumes (each) of water and dried over 0.03 parts of anhydrous sodium sulphate. The solvents were removed under reduced pressure of 50-100 Torr and temperature below 70° C., affording the 4-hyroxymyrtenyl pivalate (5) as an oil.

Step 5: Grignard synthesis of 1′,1′-dimethylheptanol from 2-octanone

To a suspension of 1.25 molar equivalents of magnesium turnings in 13.7 parts of ethyl ether, were added 0.004 equivalents of iodine while stirring. The stirring was continued until the colour of the solution faded to clear or to slightly yellow. To the resulting mixture were added 1.12 molar equivalents of iodomethane over 4-8 hours so that a gentle reflux was maintained. After another 2 hours one molar equivalent of octanone (6) was added over a period of 4-6 hours. The mixture was stirred for 2 hours at 20-25° C. then the stirring was discontinued and the mixture was allowed to settle overnight. The solution was decanted onto 2 parts of ice water, and acidified to pH 5.5-6.0 with acetic acid. The layers were separated and the aqueous phase was extracted three times with 0.33 volumes (each) of ethyl acetate. The extracts were combined, washed with 1 volume of water, 1 volume 5% sodium bicarbonate, twice with 10% sodium chloride (each) and dried over 0.1 parts of anhydrous sodium sulphate. The solvents were removed at 50° C. and 45-50 Torr. The reaction product 1,1-dimethylheptanol (7) is a colourless or pale yellow oil.

Step 6: Alkylation of 2,6-dimethoxyphenol with 1′,1′-dimethylheptanol

To a solution of 2,6-dimethoxyphenol (8) in 1.3 volumes of methansulfonic acid were added 1.1 molar equivalents of 7 and the resulting mixture was stirred under argon at 50-55° C. over a period of 30 hours and then poured onto 2.5 parts of ice water. The mixture was extracted three times with 0.5 volumes (each) of methylene chloride and the combined organic phases were washed once with 1 volume of water, once with 0.4 volume of 7% sodium bicarbonate, and twice with 1 volume of saturated aqueous solution of sodium chloride (each). The combined organic layers were dried overnight on 0.05 parts of anhydrous sodium sulphate, and the solvent was removed in vacuum at 80° C. to afford 4-(1′,1′-dimethylheptyl)-2,6-dimethoxyphenol (9) as an oil, used directly in the next step.

Step 7: Esterification of 4-(1′,1′-dimethylheptyl)-2,6-dimethoxyphenol with diethyl phosphite

To a solution of 9 in 0.5 volumes of carbon tetrachloride were added 1.5 molar equivalents of diethyl phosphite. The mixture was cooled to −10°C. and treated with 1.5 molar equivalents of triethylamine over a period of 5 hours while cooling. The mixture was then gradually warmed overnight to room temperature (20-25° C.), diluted with 2.5 volumes of methylene chloride and subsequently washed with 0.5 volumes of water, once with 0.5 volume of 0.5 N aqueous solution of 2 N NaOH, once with 0.5 volume of 0.5 N aqueous solution of hydrochloric acid and then three times with 0.25 volume of saturated aqueous solution of sodium chloride (each). The combined organic phases were dried over 0.1 part of anhydrous sodium sulphate and the solvents removed in vacuum. The resulting oil was diluted with an equal amount of petroleum ether (w/v) and crystallized at room temperature for 15-24 hours. The obtained crystals of 4-(1′,1′-dimethylheptyl)-2,6-dimethoxyphenyl diethylphosphate (10) were filtered, washed with petroleum ether and dried. An additional crop of product may be obtained by concentrating the mother liquor and recrystallizing as above.

Step 8: Reduction of 4-(1′,1′-dimethylheptyl)-2,6-dimethoxyphenyl diethylphosphate with lithium/ammonia

A solution of 10 in 2 parts ethyl ether and 0.4 parts tetrahydrofuran was added dropwise to 1.25 volumes of liquid ammonia, followed by the addition of 2.3 molar equivalents of lithium metal in small pieces at a rate to maintain a blue colour. The mixture was stirred for one hour and then poured into four volumes of 14% aqueous solution of ammonium chloride. The organic layer was separated retained and the aqueous layer was extracted three times with 0.4 volumes of methylene chloride (each). The combined organic phases were washed three times with 0.25 volumes of water (each) and dried over 0.025 parts of anhydrous magnesium sulphate. The solvents were removed in vacuum below 85° C. The resulting oil was flashed distilled under vacuum at below 200° C. to afford 1-(1′,1′-dimethylheptyl)-3,5-dimethoxybenzene (11) as an oil.

Step 9: Demethylation of 1-(1′,1′-dimethylheptyl)-3,5-dimethoxybenzene with boron tribromide

A solution of 11 in 3 volumes of methylene chloride was added dropwise to a stirred solution of 3 molar equivalents of boron tribromide in 6.7 volumes of methylene chloride, at (−15)-(−10)° C. over a period of 4-8 hours. The mixture was gradually warmed overnight to room temperature (20-25° C.) and 1 volume of ice water was added. The organic phase was separated and retained and the aqueous phase was extracted twice with 0.3 volumes of methylene chloride. The organic phases were combined, dried over 0.05 parts anhydrous magnesium sulphate, and the solvent was removed in vacuum below 85° C. The residue was refluxed with five volumes of hexane, cooled to 20-25° C. and the resulting crystals of dimethylheptyl resorcinol (12) filtered off, rinsed with hexane and dried under vacuum at 50-55° C.

Step 10: Coupling of 4-hydroxy myrtenyl pivalate with 5-(1′,1′-dimethylheptyl)-resorcinol

To a mixture of 1.1 molar equivalents of 5 and 1.0 molar equivalent of 12 in 24 volumes of methylene chloride were added four molar equivalents of boron trifluoride etherate, at (−15)-(−10)° C. over one hour. The reaction mixture was maintained at the above temperature for 2.5 hours, then treated with another four molar equivalents of boron trifluoride etherate over one hour and stirred at the same temperature for another 2.5 hours. The reaction mixture was poured onto 0.5 parts of crushed ice containing 29 molar equivalents of sodium bicarbonate and left overnight at 20-25° C. The organic layer was separated and washed 3 times with 1.4 volumes (each) of 5% aqueous solution of sodium bicarbonate and dried over 0.05 parts of anhydrous sodium sulphate. The solvent was removed in vacuum at 50 Torr and 45° C. The residue was passed through 10 parts of silica gel 60-230 mesh using toluene as eluent. The fractions containing dexanabinol pivalate were collected and the solvent was removed in vacuum to afford dexanabinol pivalate (13) as an oil.

Step 11: Hydrolysis of dexanabinol pivalate to dexanabinol

To a solution of 13 in 10 volumes of tetrahydrofuran were added 4.3 molar equivalents of 1.0 M lithium aluminum hydride in tetrahydrofuran at (−10)−5° C. over 3-5 hours. The reaction mixture was stirred for one hour at 20-25° C. then cooled to 5° C. and treated dropwise with 0.1 5 volumes of ethyl acetate, while maintaining the temperature below 5° C. To the reaction mixture were added 0.5 part crushed ice and 1 part water, and the mixture was acidified to pH 4.0 with about 0.5 volume of acetic acid and then extracted with six times (each) with 0.1 volume of a mixture of hexane: ethyl acetate (2:1). The combined extracts were washed 3 times with 0.25 volumes (each) of water and 3 times with 0.3 volume (each) of 5% aqueous solution of sodium bicarbonate and then dried over 0.5 parts of anhydrous sodium sulphate. The solvents were removed in vacuum at 50 Torr and 40° C. and the residue was recrystallized from 6 volumes of acetonitrile brought to temperature near reflux at 70-81.6° C. The white crystals of dexanabinol (14) were filtered, rinsed with cold acetonitrile (2-8° C.) and dried in a vacuum oven at 60° C. for three hours. The resulting dexanabinol was recrystallized from 28 parts 1:1.2 water:ethanol, filtered, and dried to constant weight at 65-75° C. and 1-5 Torr.

As above stated, the crystallization performed at the final step and the nature of the solvent used for this purpose are crucial for the purity of dexanabinol. Previously disclosed procedures for the synthesis of dexanabinol (U.S. Pat. No. 4,876,276) did not teach or suggest the importance of the final crystallization step in achieving the enantiomeric purity required for pharmaceutical or clinical grade material. Moreover, it is now disclosed that the selection of solvent or mother liquor for the final crystallization may affect the purity of the product, as well as the efficiency of the crystallization.

The active pharmaceutical ingredient following crystallization from acetonitrile is superior to that recovered from any previously published procedure, both in terms of enantiomeric purity and overall yield.

The above process is highly reproducible, as will be shown below in Table 2, and was performed successfully for the preparation of multiple batches of 100 to several hundred grams of dexanabinol. The process was performed under cGMP (current Good Manufacturing Practice) conditions. To the best of our knowledge, dexanabinol was prepared till then in laboratory scale not exceeding few grams and the successful scaling up of the process has important implications regarding the feasibility of the preparation of dexanabinol in scales more appropriate to its clinical testing.

The efficiency of acetonitrile crystallization was later confirmed in a controlled experiment where HU-211 was mixed with a high amount of HU-210 impurity, 90:10 respectively, simulating an elevated level of contamination. The ratio between the two enantiomers was determined by chiral HPLC exemplified hereinbelow and found to drop from 88.6:11.4 on mixing to 100.0:0.0 after crystallization with acetonitrile. Even if it cannot be assured that HU-210 was totally eliminated by this process, its level was dramatically reduced by at least four orders of magnitude to below level of detection.

Since this initial finding, acetonitrile was used for the enantiomeric separation by crystallization in all large scale synthesis and repeatedly yielded dexanabinol of high enantiomeric purity. Since no comparative crystallization experiments were performed on dexanabinol, the existence of additional solvents or mixture of solvents that could be efficient in the enantiomer separation of HU-211 from HU-210 cannot be ruled out.

The purity can be increased, if necessary, by repeating the crystallization step with acetonitrile. Such an approach was successfully performed once with clinical scale batch 00139 prepared according to the procedure described in Example 2. After single acetonitrile crystallization the product did not comply with the specification with an HU-211 content of less than 94.7%. Following additional recrystallization with acetonitrile the HU-211 content raised by at least 4.5% to 99.2%, the content of HU-210 was then determined and the enantiomeric excess and absolute enantiomeric amount of dexanabinol were calculated to be 99.96% and 99.98% respectively. For comparison, the final crystallization step with ethanol:heptane only slightly further improved the optical purity of the product with a final HU-211 content of 99.4%, an enantiomeric excess of 99.98% and an absolute enantiomeric amount of 99.99%. Additional round(s) of recrystallization are performed if the initial purity of dexanabinol is not adequate and does not fall within the specifications. The ultimate recrystallization step using other solvents is not aimed at further increasing the enantiomeric purity, but mainly at removing traces of acetonitrile.

### Example 2

### Large Scale Preparation of Dexanabinol of High Enantiomeric Purity

An alternative process was developed for the preparation of large scale batches in the kilogram range, and two batches of 1.8 and 2.6 kg of dexanabinol were successfully prepared as will be shown in Table 2 below.

The large scale synthetic process differs from the process described in Example 1 at specific steps and the modifications are as follows. In the early stages of the process the changes include modifications in distillation conditions or in solvents. In step 2, the crude myrtenyl pivalate previously used for the subsequent step without further purification, was now further distilled under high vacuum at 2 Torr up to 180° C. Under such conditions, the distillate contained at least 80% myrtenyl pivalate (3) with 53% yield. In step 3, the crude 4-oxomyrtenyl pivalate is further distilled at higher temperature up to 190° C. under high vacuum at 1 Torr, instead of previous 120-165° C. and 0.1-0.15 Torr. The distillate was diluted with two volumes of n-hexane instead of previous n-pentane. In step 4, the mixture of 4-oxomyrtenyl pivalate with sodium borohydride was extracted with 2.5 volumes of dichloromethane (DCM) instead of previous hexane. The solvent methanol/DCM was removed under reduced pressure at 50-100 Torr and temperature below 70° C. Then 1 volume of DCM was added to afford the 4-hyroxymyrtenyl pivalate (5) in DCM solution in yield of about 84.5%. The modifications introduced in the later stages of the process being more extensive, the synthetic steps will be described in their entirety.

Step 5: Grignard synthesis of 1′,1′-dimethylheptanol from 2-octanone

A 1 liter reactor under N₂ atmosphere, was filled with 468.3 g methyl magnesium chloride 23% solution in tetrahydrofuran (THF) (1.2 eq.) and 122 ml of THF. Then 153.85 g of 2-octanone (6) (1.2 mole) were added at 20-25° C. during 90 minutes. The reaction mixture was then stirred for 24 hours at room temperature, while monitoring the reaction progress by gaz chromatography. The reaction mixture was then transferred to a second 1 liter reactor containing 154 ml of water, while keeping the temperature under 20° C. The reaction mixture was then passed through frit glass in order to eliminate mineral salts of magnesium. A 320 g of 4% solution of NaCl was added to the filtrate and 154 ml of methyl tert butyl ether (MTBE). The so obtained mixture was stirred for 10 min at 20° C. and then the organic phase was decantated. The aqueous phase was extracted with a second portion of 154 ml of MTBE. The combined organic phases were washed with 154 ml of water. The solvents were then removed by distillation at initial mass temperature of 63° C. and final mass temperature of 93° C. for 7 hours, until orange liquid residue was obtained. The residue was cooled to room temperature and 77 ml of toluene were added. The mixture was heated at atmospheric pressure up to distillation of toluene (117-120° C.) then the reaction mass was cooled to room temperature obtaining 153 g of the product (7) at a concentration of about 70% in toluenic solution (1.06 mole) 88.5% yield.

Step 6: Alkylation of 2,6-dimethoxyphenol with 1′,140 -dimethylheptanol

To a solution of 2,6-dimethoxyphenol (8) in 1.3 volumes of methansulfonic acid were added 1.1 molar equivalents of 7 in toluene and the resulting mixture was stirred under argon at 50-55° C. over a period of 30 hours and then poured onto 2.5 parts of ice water. The mixture was extracted three times with 0.5 volumes (each) of methylene chloride and the combined organic phases were washed once with 1 volume of water, once with 0.4 volume of 7% sodium bicarbonate, and twice with 1 volume of saturated aqueous solution of sodium chloride (each). The combined organic layers were dried overnight on 0.05 parts of anhydrous sodium sulphate, and the solvents were removed in vacuum at 80° C. to afford 4-(1′,1′-dimethylheptyl)-2,6-dimethoxyphenol (9) as an oil, used directly in the next step.

Step 7: Esterification of 4-(1′,1′-dimethylheptyl)-2,6-dimethoxyphenol with diethylchlorophosphate

To a 150 ml reactor, 0.3 g of dimethylamino-4-pyridine and 136 g of the crude product 9 (0.486 mole) were added in 100 g of DCM. The reaction mixture was cooled to about 0° C., 109 g of diethylchlorophosphate were added. While maintaining the temperature at 10° C., 64 g of triethylamine were added over a period of 1 hour. The mixture was then gradually warmed overnight to room temperature (20-25° C.), diluted with 204 ml of toluene and subsequently washed with 7% solution of NaCl. The aqueous phase was discharged and the organic phase washed with 68 ml of water, and again the aqueous phase was eliminated (pH=1). The reaction mixture was heated to 85° C. under atmospheric pressure to eliminate the solvents (DCM/Toluene/water) and then under reduce pressure to complete distillation. The resulting brown solution was cooled to 60° C. and 178 ml of heptane were added. The mixture was cooled until crystallization was obtained at 36° C., then the solution was further cooled down to 10° C. stirred at that temperature for 1 hour and then filtered. 184 g of dried product (10) were obtained (0.442 mole) 91% reaction yield.

Step 8: Reduction of 4-(1′,1′-dimethylheptyl)-2,6-dimethoxyphenyl diethylphosphate with lithium/ammonia

A 1 liter reactor previously cooled at −70° C. was charged with 375 ml of liquid ammonia. Then at a temperature under −50° C., 6.25 g of lithium metal were added. Then, the obtained blue suspension was cooled to −70° C. and during 2 hr a previously prepared solution of 124 g of product (10) (0.3 mole) in 50 ml THF and 250 ml of butyl methyl ether were added. After the addition, the reaction mixture was stirred for an additional hour. At the end of the reaction 25 g of ammonium chloride were carefully added portion wise. The temperature of the resulting light brown solution was slowly increased up to 20° C. Then 375 ml of water were added, which led to ammonia evolution. The reaction mixture was heated up to 85° C. under atmospheric pressure to eliminate ammonia and part of THF/MTBE. Then the reaction mixture was cooled down to room temperature and 375 ml of water and 500 ml of toluene were added. The aqueous phase was then discharged and the toluene phase was washed with 250 ml of water, and again the aqueous phase was discharged. The reaction mixture was then heated up to reflux to remove under atmospheric pressure water and part of the toluene to obtain a 224.5 g of a toluene solution containing about 31% of the product (11) (0.262 mole), about 87% yield.

Step 9: Demethylation of 1-(1′,1′-dimethylheptyl)-3,5-dimethoxybenzene with boron tribromide

A solution of 11 in 3 volumes of toluene was added dropwise to a stirred solution of 3 molar equivalents of boron tribromide in 4 volumes of toluene, at (−15)-(−10)° C. over a period of 4-8 hours. The mixture was gradually warmed to room temperature (20-25° C.) over a period of about 2 hours, and then 1 volume of ice water was added. The organic phase was separated and retained and the aqueous phase was extracted twice with 0.3 volumes of toluene. The organic phases were combined, dried over 0.05 parts anhydrous magnesium sulphate, and the solvent was removed in vacuum below 85° C. The residue was refluxed with five volumes of heptane, cooled to 20-25° C. and the resulting crystals of dimethylheptyl resorcinol (12) filtered off, rinsed with hexane and dried under vacuum at 50-55° C.

Step 10: Coupling of 4-hydroxy myrtenyl pivalate with 5-(1′,1′-dimethylheptyl)-resorcinol

A 0.5 liter reactor previously filled with nitrogen, was charged with 25.25 g of 5 (0.1 mole) and 36.3 g of 12 (0.14 mole) in 247 ml of DCM. The reaction mixture was cooled to (−15)-(−20)° C. under stirring and while keeping the temperature below −14° C. 42.6 g of boron trifluoride etherate were added. The resulting brownish solution was maintained at −15° C. for at least 1 hr. When the reaction was completed, a previously prepared solution of 15.15 g of sodium bicarbonate in 288 ml of water was added while letting the temperature rise up to 20° C. Then the two phases were separated. The organic phase was washed again with sodium bicarbonate solution and again phases were separated. To the organic phase 76 ml of water were added and then 40 g of sodium hydroxide 30.5% solution. After 10 minutes of stirring the two phases were separated. The organic phase was washed with 100 ml of water and again phases were separated. Then the organic phase was acidified with hydrochloric acid at 15-20° C. until pH 4-4.5 and the phases were separated. The organic phase was washed with 100 ml of water and then phases were separated. The solvent was removed under reduce pressure at 40-50° C. The oily residue was diluted with 150 ml of THF. The solution obtained was cooled to 20° C. The product (13) was not further isolated and it was used in the next step as a solution in THF.

Step 11: Hydrolysis of dexanabinol pivalate to dexanabinol

A 2 liters reactor was filled with 780 g of 12% solution of 13 (0.2 mole) and cooled down to 0-(−5)° C. Then 359 g of LiAlH₄ 1M solution in THF were added and the reaction mixture was stirred at that temperature for 1 hour. Then 195 ml of ethyl acetate were added and while stirring vigorously 1200 ml of water were added. The reaction mixture was warmed to 25° C. and 75 g of hydrochloric acid 37% were added. Then the two phases were separated. Adding 270 ml of 5% solution of sodium bicarbonate neutralized the organic phase, and then the aqueous phase was eliminated. The organic phase was washed with 200 ml of water and the water phase was eliminated. The solvents from the organic phase were removed under vacuum 50 Torr at 40-50° C. The residue was recrystallized from 6 volumes of acetonitrile brought to temperature of about 90° C. to remove residual solvents. Then the reaction mixture was allowed to cool until the beginning of the precipitation. The temperature was maintained for 1 hour at 0-5° C. and the white crystals of dexanabinol (14) were filtered, rinsed with cold acetonitrile (2-8° C.) and dried in a vacuum oven at 60° C. for three hours. The resulting dexanabinol was recrystallized from ethanol:heptane 3:5, filtered, and dried to constant weight at 65-75° C. and 1-5 Torr. The pivotal crystallization step is performed with acetonitrile, which is removed by recrystallization from ethanol:heptane instead of previously used water:ethanol. As previously explained, the enantiomeric purity can be further increased, if necessary, by repeating the crystallization step with acetonitrile

The main advantages of the process of Example 2 over Example 1 lie in the utilization of solvents appropriate to industrial large-scale synthesis and in the adaptation or elimination of certain isolation and purification steps enabling a simplified continuous process. The new process has allowed the preparation of batches of kilogram quantities, to suit commercial production of the drug.

### Example 3

### Characterization of Dexanabinol Enantiomeric Purity

Certain current specifications for dexanabinol drug substance are listed in Table 1. The abbreviations used in this table means: IR infrared, UV ultraviolet, ppm parts per million, EU endotoxin unit, CFU colony forming unit, HPLC high pressure liquid chromatography, TLC thin layer chromatography; and the percentages are expressed as weight per weight (w/w).

Unless otherwise stated, the characterization is performed using classical validated analytical methods following established standard operating procedures. When appropriate, samples are compared to reference materials, which are predetermined set standards that may themselves be ultrapure standards. HU-211 and HU-210 reference material were prepared by additional crystallization steps and chromatographic separations. Compounds that serve as reference undergo thorough analyses, which includes, on top of the assay listed in Table 1, nuclear magnetic resonance (NMR), Mass spectra (MS) and element analysis. Per definition these ultrapure compounds will be referred to as 100%. The reference material for HU-211 was prepared in-house, while the reference material for HU-210 was purchased from Tocris.

TABLE 1
Specifications for dexanabinol of high enantiomeric purity.

| Test | Specification |
|---|---|
| Appearance | White to off-white solid |
| Identification | |
| by IR | IR spectrum exhibits maxima and |
| | minima at the same wavelengths |
| | as the reference material |
| by UV | UV spectrum exhibits maxima and |
| | minima at the same wavelengths |
| | as the reference material |
| HU-211 content | Not less than 98.0% |
| (Reversed phase HPLC) | |
| HU-210 content (Chiral HPLC) | Not more than 0.05% |
| Melting Point Range | 140-143° C. |
| Water | Not more than 0.1% |
| Loss on Drying | Not more than 0.5% |
| Specific Rotation | +220 ± 10° |
| | (0.1% w/v in chloroform |
| | at 25° C., at 589 nm) |
| Bacterial endotoxins | Not more than 1.5 EU/mg |
| Total aerobic microbial count | Not more than 10 CFU/g |

All clinical grade, intermediate scale, batches of dexanabinol prepared to date were tested for these characteristics and were shown to conform to the specifications. As previously explained one of the most important issues regarding the analysis of dexanabinol is the content of the psychoactive enantiomer HU-210. The determination of the chiral purity is performed using HPLC methodology modified from Levin et al. (Levin S. et al., J. Chromatography A, 654: 53-64, 1993). Briefly, one set of calibration standard solutions was prepared using the HU-210 reference material diluted into HPLC mobile phase to yield standards of 0.125 to 3 μg/ml. Similarly, the sample was dissolved into the mobile phase to yield a solution of 5 mg/ml. The mobile phase is composed of 96% volume/volume (v/v) of n-hexane and 4% v/v of isopropanol, each HPLC grade and previously filtered through a 0.45 μm nylon membrane, the mixture was degassed using a sonication bath for a few seconds. The HPLC is performed on a chemically modified amylose-based chiral column ChiralPak AD-H, 250×4.6 mm, 5 μm particle size (Daicel Ltd). The chiral stationary phase is a tris(3,5-dimethylphenylcarbamate) derivative of amylose immobilized on macroporous silica gel. The flow rate is 1 ml per minute, the chromatography is performed at ambient temperature of about 25° C. and the detection is performed at 215 nm. The controls or samples are injected at a volume of 40 μl and a run is performed for 50 minutes. The HPLC mobile phase is injected first as a blank, then the 50 μg/ml standard of HU-210 mixed with HU-211 to determine the retention time for each enantiomer and confirm the separation of the peaks and thus the efficiency of the analytical method. HU-210 elutes after HU-211 with a typical relative retention time of about 1.4. Then the 0.125 to 3 μg/ml calibration solutions are injected and a regression analysis on the response peak versus concentration is performed, the correlation coefficient R-square must be above 0.98. The sample, prepared in duplicates, is then injected and the analyte peak is integrated and the concentration of the HU-210 impurity is determined from the calibration curve. The presence or absence of HU-210 is reconfirmed by injection of a confirmation sample prepared by spiking the original sample with 0.02% HU-210. This method was thoroughly validated for selectivity, precision, linearity, accuracy and robustness. There is no interference with sample blank or with dexanabinol related compounds, such as dexanabinol pivalate (13 in Scheme 5). Quantitation of HU-210 is linear at least within the range of 0.0025 up to 0.12% w/w of dexanabinol. The detection and quantitation limits of HU-210 are respectively 0.00125 and 0.0025% w/w of dexanabinol. The method is highly repeatable as measured by low relative standard deviation (RSD) when the same sample is injected six times (system repeatability RSD<2%), when six replicates are injected (method repeatability RSD<7%) and when 6 replicates are tested on two HPLC systems (intermediate precision˜5%). This method allows to determine the level of HU-210 in the dexanabinol drug substance sample with accuracy and thus the level of enantiomeric purity of HU-211, as expressed as enantiomeric excess over HU-210, with confidence.

The adaptations brought to the method of Levin et al. include: the use of a single shorter wavelength of detection, namely 215 nm instead of the previous double simultaneous detection at 220 and 270 nm; the utilization of smaller particles, ≦5 μm instead of 10 μm; modification of the sample loading conditions with an increase in injection volume, namely 40 μl instead of 20 μl; and, in sample concentration with 5 mg/ml instead of previous 0.1 mg/ml. These modifications together lead to a significant improvement of over 30-fold in the lower limit for reliable quantitation of the (3R,4R) enantiomer in term of concentration. Thus, with the present analytical methods HU-210 can be detected at a concentration of 0.125 μg/ml (corresponding to an amount as low as 5 ng per sample), instead of the previous estimate of 3.9 μg/ml. The lower limit for detection of HU-210 achieved by the present method allows confident determination of higher enantiomeric excess than previously possible.

Levin et al. analyzed 2 μg of 4-oxomyrtenyl pivalate and estimated the limits of determination to be 60 ng, indicating that the enantiomeric excess of this intermediate could be determined with certainty only if it falls below 95%. According to the methods of the present invention, when analyzing 200 μg of dexanabinol the limit of detection of HU-210 is 5 ng, which allows determination of enantiomeric excess above 99.99%.

Similarly, the amount of HU-211 in dexanabinol drug substance is assayed by reversed phase (RP)-HPLC. The HPLC column used is a Hypersil BDS RP-18 3 μm, 150×4.6 mm, maintained at 30° C. The mobile phase is composed of 60% acetonitrile and 40% 10 mM ammonium acetate buffer pH 5.2. The injection volume is 15 μl, the flow rate is 1.2 ml per minute, detection is performed at 280 nm and a run lasts 45 minutes. Sample or HU-211 reference standard are dissolved in acetonitrile, mixed by vortex and sonicated to complete dissolution to yield solutions of 1 mg/ml. Acetonitrile is injected as blank, followed by five injections of the standard solution to ensure that the RSD is below 2.0%. The retention time of dexanabinol is about 23 minutes under those conditions. The sample to be assayed is prepared in duplicate and is then injected. The percent of HU-211 is then calculated using the following formula % HU-211=(R_{U}/R_{S})×(W_{S}/V_{S})×(V_{U}/W_{U})×100, wherein R_{U} and R_{S} are the peak responses of the unknown sample and standard respectively, W_{U} and W_{S} are the weights (in mg) and V_{U} and V_{S} are the volumes (in ml) of the unknown sample and standard respectively.

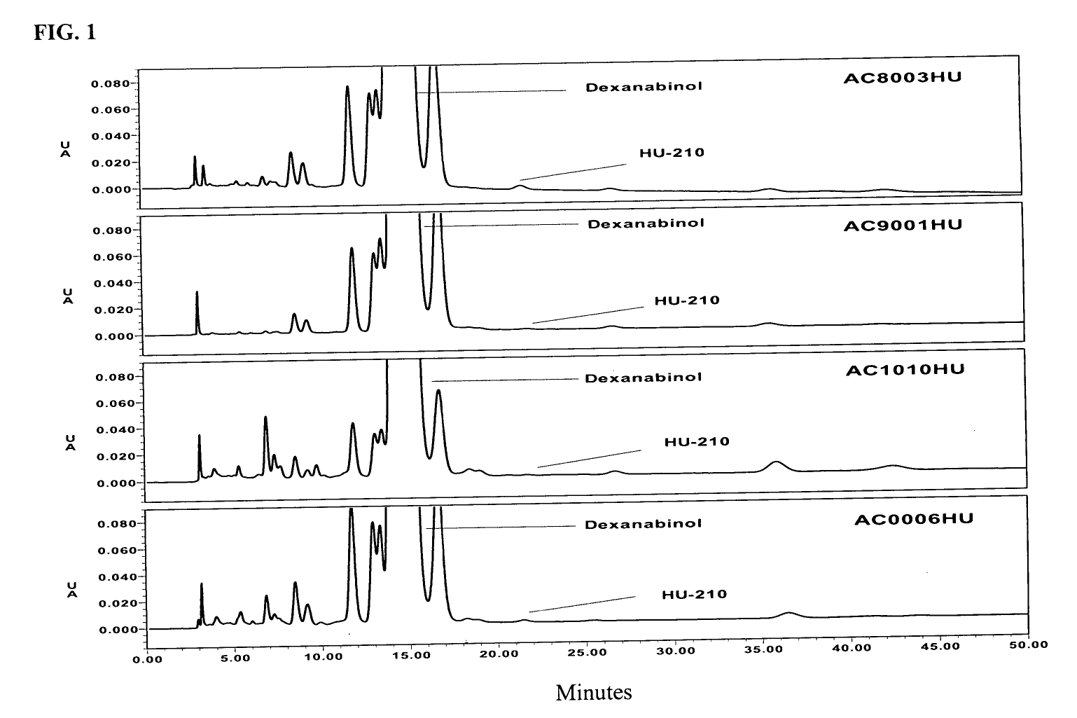

Using the above-described methods for quantitation of HU-210 and HU-211, the enantiomeric excess of dexanabinol was determined in six clinical grade batches of the drug substance. These batches of active pharmaceutical ingredient (API) were later used for the preparation of drug product as used in the clinical trials. Chromatograms of the HPLC analysis of four of the batches, wherein the absorbance units (AU) are plotted against retention time, are displayed in FIG. 1. All other parameters were found conform to specifications and met the acceptance criteria. The results regarding the optical purity, expressed either as enantiomeric excess or as absolute enantiomeric amount of dexanabinol, are shown in Table 2.

TABLE 2
Contents of HU-211 and HU-210 in six large scale batches.

| | | | HU-210 | | Absolute |
|---|---|---|---|---|---|
| | Amount | HU-211 | (% | Enantiomeric | enantiomeric |
| Batch | (g) | (% w/w) | w/w) | excess (%) | amount (%) |
| AC8003HU | 103 | 98.80 | 0.0160 | 99.97 | 99.98 |
| AC9001HU | 235 | 98.80 | 0.0036 | 99.99 | 100.00 |
| AC0006HU | 213 | 98.70 | 0.0079 | 99.98 | 99.98 |
| AC1010HU | 392 | 99.10 | 0.0025 | 99.99 | 99.99 |
| 00139 | 2635 | 99.40 | 0.0110 | 99.98 | 99.99 |
| 00175 | 1750 | 98.50 | 0.0150 | 99.97 | 99.98 |

It can be deduced from Table 2, that the synthetic procedures previously described in Examples 1 and 2 are suitable for the preparation of clinical grade batches of dexanabinol of very high optical purity as expressed by an enantiomeric excess of at least 99.90% and an absolute enantiomeric amount of at least 99.95%.

### Example 4

### Formulation of Dexanabinol of High Enantiomeric Purity for Clinical Use

Dexanabinol is an extremely lipophilic compound with a computed Log P of 7.69 (Advanced Chemistry Development, software Ver. 4, by ACD labs.) and an experimental Log P of 7.44 (Thomas B. F. et al., J. Pharmacol. Exp. Ther. 255: 624-30, 1990) rendering it essentially insoluble in water (calculated water solubility 0.1 ng/ml). Though dexanabinol can be formulated in a variety of compositions that accommodate its lipophilic nature, the clinical trials are performed with the drug substance in the following formulation wherein all ingredients are of pharmacopeal grade. Dexanabinol drug substance is formulated as a 5% w/v concentrate in a cosolvent vehicle composed of CREMOPHOR EL® (polyoxyl 35 castor oil; 65% w/v) and absolute ethanol (26.5% w/v). The dexanabinol cosolvent concentrate also contains 0.01% w/v edetic acid and 0.5% w/v Vitamin E (DL-α-tocopherol) as antioxidants. This parenteral 5% cosolvent solution is a clear, slightly yellow, sterile and pyrogen-free concentrate of dexanabinol for injection which must be diluted prior to intravenous infusion 1/20 to 1/100 with sterile 0.9% sodium chloride solution for injection. The drug product is preservative-free and sterilization is achieved via a sterile filtration and aseptic processing technology. The quantitative composition of the 5% dexanabinol parenteral cosolvent concentrate is given in Table 3. The dexanabinol drug substance is manufactured as previously described in Example 1 or 2 and according to the specifications in Example 3, specifically in enantiomeric excess of at least 99.90% and of absolute enantiomeric amount of at least 99.95%. All the inactive ingredients used, ethanol absolute, edetic acid, Vitamin E and CREMOPHOR EL®, are manufactured according to standards set in the British Pharmacopea, United States Pharmacopea or European Pharmacopea, all being considered acceptable.

As previously stated the parenteral concentrate formulation has to be diluted prior administration. In a stability study, the above-described clinical formulation of dexanabinol of high enantiomeric purity was diluted with sterile 0.9% sodium chloride solution for injection at a ratio of 1:5 up to 1:500. The ready for injection diluted drug concentrate were stable at all dilution ratios for up to 24 hours as determined by HPLC analysis performed on filtrates collected at predetermined time points along the duration of the study.

TABLE 3
Composition of dexanabinol parenteral concentrate.

| Ingredient | mg/ml | mg/g |
|---|---|---|
| Dexanabinol | 50.0 | 51.5 |
| Ethanol Absolute | 265.0 | 273.2 |
| CREMOPHOR EL ® | 650.0 | 670.0 |
| Edetic Acid | 0.1 | 0.1 |
| Vitamin E | 5.0 | 5.2 |

### Example 5

### Other Pharmaceutical Compositions for Dexanabinol of High Enantiomeric Purity

The above described pharmaceutical composition in use in clinical trial for dexanabinol of high enantiomeric purity has been selected following intensive formulation development. It is well known that cosolvents are employed in various FDA approved parenteral products. Drugs dissolved in these cosolvents are usually prepared as concentrated solutions that are diluted with sterile sodium chloride or dextrose solutions before injection. A variety of non-aqueous vehicles have been used successfully as cosolvents for the solubilization and intravenous delivery of many poorly soluble drugs. A survey of FDA-approved parenteral products shows five water-miscible cosolvents as components of sterile formulations: glycerin, ethanol, propylene glycol (PG), polyethylene glycol (PEG), and dimethylacetamide. Other non-aqueous vehicles include surface-active agents such as TWEEN® 80 and CREMOPHOR EL®. Surfactant agents are usually incorporated into parenteral preparations to provide an increase in drug solubility through micellization and to prevent drug precipitation upon dilution. The vehicle of choice should provide for adequate stability, have an acceptable safety profile and allow for drug administration within the shortest period of time leading to the highest possible plasma concentration Cmax thereby providing for the maximum achievable therapeutic drug concentrations in the target organ with minimal administration risks.

Cosolvent formulations.

The goal of this study was to find a suitable cosolvent formulation for a concentrate of dexanabinol of high enantiomeric purity to be diluted with sterile saline solution before injection. The compositions of the cosolvent concentrate formulations tested are presented in Table 4. All formulations contained 1% dexanabinol and compositions of FDA-approved cosolvent vehicles. The concentrations of the various ingredients are expressed as % weight/weight.

TABLE 4
Compositions of various cosolvent formulations.

| FORMULATION | CREMOPHOR | PEG | | TWEEN ® | Benzyl | | | Drug |
|---|---|---|---|---|---|---|---|---|
| NUMBER | EL ® | 300 | Ethanol | 80 | Alcohol | PG | H₂O | dissolution |
| SA 46-4 | | 65 | 24 | 8 | 3 | | | soluble |
| SA 46-5 | | 66 | 26 | 8 | | | | soluble |
| SA 46-13 | | | | 4 | | 20 | 76.0 | insoluble |
| SA 46-14-1 | | 50 | | | | | 50.0 | insoluble |
| SA 46-14-3 | 11.5 | | | | | | 88.5 | insoluble |
| SA 46-15-1 | 7 | | | | | | 93.0 | insoluble |
| SA 46-15-2 | 70 | | 30 | | | | | soluble |
| ED 61 48-1 | | | 10 | | | 40 | 50.0 | insoluble |

As can be seen from Table 4, only anhydrous formulations SA 46-4, SA 46-5 and SA 46-15-2 containing surfactants (TWEEN® 80 or CREMOPHOR EL®) were able to dissolve dexanabinol of high enantiomeric purity. In cosolvent mixtures containing water, the drug was insoluble, but aqueous cosolvents are certainly appropriate for less lipophilic prodrugs, salts or esters of dexanabinol.

CREMOPHOR EL®:ethanol formulations.

Once it was established that a cosolvent formulation made of CREMOPHOR EL® (polyoxyl 35 castor oil) and ethanol is appropriate to dissolve the drug, a matrix of such formulations was prepared at various concentrations (from 30 to 70% w/w of each ingredient) and with increasing amounts of dexanabinol of high enantiomeric purity (20, 50 and 100 mg/ml). The exact composition of these formulations is described in the left hand side of Table 5. The drug cosolvent concentrates were diluted at various ratios in saline and the stability of the drug in the resulting solutions was monitored for 24 hours. The results are detailed in the right hand side of Table 5.

TABLE 5
Compositions and post-dilution stability of CREMOPHOR EL ®:ethanol
formulations.

| | Cosolvent Composition | | Stability following dilution in saline | | |
|---|---|---|---|---|---|
| Dexanabinol | % CREMOPHOR | % | Dilution | Dilution | Dilution |
| (mg/ml) | EL ® | Ethanol | 1/5 | 1/10 | 1/20 |
| 20 | 70 | 30 | Stable | Stable | Stable |
| | 50 | 50 | Stable | Stable | Stable |
| | 30 | 70 | Stable | Stable | Stable |
| 50 | 70 | 30 | Stable at | Stable at | Stable at least |
| | | | least 7 hours | least 7 hours | 7 hours |
| | 50 | 50 | Stable at | Stable at | Stable at least |
| | | | least 7 hours | least 4 hours | 7 hours |
| | 30 | 70 | Crystals | Crystals | Crystals |
| | | | appeared at 2 | appeared at | appeared at |
| | | | hours | 2 hours | 2.5 hours |
| 100 | 70 | 30 | Crystals | Crystals | Crystals |
| | | | appeared at 2 | appeared at | appeared at |
| | | | hours | 2 hours | 3.5 hours |
| | 50 | 50 | Crystals | Crystals | Crystals |
| | | | appeared at | appeared at | appeared at |
| | | | 1.5 hours | 1.5 hours | 1.5 hours |
| | 30 | 70 | Crystals | Crystals | Crystals |
| | | | appeared at | appeared at | appeared at |
| | | | 45 minutes | 55 minutes | 1.5 hours |

The results obtained with these nine formulations showed that CREMOPHOR EL®:ethanol consolvent formulations were able to successfully dissolve up to at least 100 mg/ml of dexanabinol of high enantiomeric purity. The higher the amount of CREMOPHOR EL® the more stable the drug after dilution of the cosolvent concentrate into aqueous solutions. The 70:30 CREMOPHOR EL®:ethanol formulation was selected as a basis for further optimization. Having the clinical application in mind where the cosolvent concentrate is diluted in physiological buffer immediately prior to injection, the 50 mg/ml dose was selected for further studies since at this concentration the diluted drug is stable for at least seven hours and the concentration allows for the injection over a short period of time. The selected formulation of 50 mg/ml dexanabinol of high enantiomeric purity dissolved in 70:30 CREMOPHOR EL®:ethanol was stable after dilution with saline at all ratios tested from 1:5 to 1:20. A dilution of 1:5 is about the minimum required prior to injection, since it is recommended not to inject solutions containing more than 10% ethanol. As already noted the final clinical formulation was shown to be stable for 24 hours in dilutions from 1:5 up to 1:500.

### Example 6

### Pharmacokinetic Studies Performed with Dexanabinol of High Enantiomeric Purity

The pharmacokinetics of dexanabinol of high enantiomeric purity formulated in CREMOPHOR EL®:ethanol as described in Example 4 were investigated in rats, rabbits, and monkeys following intravenous administration of single doses, and 14 and 28 days of repeated dosing. Human pharmacokinetics was studied during Phase I and Phase II clinical studies. Dexanabinol used in the pharmacokinetic studies was formulated as drug concentrates of 50 and 100 mg/ml and diluted with sterile 0.9% NaCl solution prior to intravenous (i.v.) administration to the desired final doses. Determination of dexanabinol concentrations in plasma and brain extracts was carried out using a validated Gas Chromatography-Mass Spectra (GC-MS) assay following solid phase extraction of the drug and derivatization. The limit of quantitation of the assay is 0.1 ng/ml.

Pharmacokinetic parameters were estimated by a non-compartmental method using WinNonlin Professional version 3.2 (Pharsight Corp., Mountain View, Calif.). The maximum plasma concentration (Cmax), when the drug is administered by infusion, is the concentration at the end of infusion. The Cmax following intravenous bolus administration is the value estimated by the software to be the concentration at t=0. The terminal slope (X) was estimated by linear regression through the last time points and used to calculate the terminal half-life (t_{1/2}) from the following equation:
t_{1/2}=0.693/λ
The area under the curve from time of dosing through the last time point (AUC_{z}) was calculated by the linear trapezoid method. The AUC extrapolated to infinity (AUC_{∞}) was calculated from the following equation:
AUC_{∞}=AUC_{z}+C_{z}/λ
where C_{z} is the concentration at the last time point predicted by the linear regression. AUC∞ was normalized for dose (mg/kg) and presented as AUC_{∞}/Dose. Mean residence time (MRT), when the drug is administered by infusion is described by the following equation:
MRT=(AUMC/AUC)−(TI)/2
Mean residence time after i.v. bolus administration is described by the following equation:
MRT=(AUMC/AUC)
where AUMC is the area under the first moment curve and TI is the length of infusion. Plasma clearance (CL), and the apparent volume of distribution at steady state (Vₛₛ) were calculated from the following equations:
CL=Dose/AUC_{∞}
Vₛₛ=MRT×CL

Brain pharmacokinetic parameters in animal studies were estimated by non-compartmental methods similar to those used for plasma data with the addition of an estimate of the time of maximum concentration (Tmax) which was assumed to be zero for the plasma data. Cmax is the concentration corresponding to Tmax. AUC_{z} and AUC_{∞} were calculated as described above. Kp, brain-to-plasma partition coefficient, was determined by means of the area method and using the equation:
Kp=AUC_{∞} brain/AUC_{∞} plasma

The percentage of oral bioavailability was calculated using the following equation:
% F=[AUCₒᵣₐₗ/Doseₒᵣₐₗ]/[AUC_{IV}/Dose_{IV}]
Non-human Pharmacokinetic Studies.

In order to support the i.v. testing of dexanabinol of high enantiomeric purity in humans, a series of acute single-dose and sub-chronic multiple dose toxicology studies were conducted to establish the safety profile of the compound in rats, rabbits, and monkeys. The 2-week and 4-week multidose studies included complete clinical and morphological evaluations. In vitro/in vivo mutagenicity studies and special toxicological evaluations have also been carried out to evaluate the safety profile of dexanabinol. The toxicology studies employed doses that were multiples of the proposed clinical doses. The results of the toxicological studies performed with dexanabinol of high enantiomeric purity indicate that the drug when formulated in CREMOPHOR EL®:ethanol as described in Example 4 is generally well tolerated following single and/or multiple i.v. doses in rats, rabbits and monkeys.

Single dose toxicity studies showed a no observed adverse effect level (NOAEL) of 50 mg/kg in Sprague Dawley rats, 25 mg/kg in New Zealand White rabbits, and 50 mg/kg in Cynomolgus monkeys. Table 6 summarizes the maximum plasma concentration (Cmax) and the area under the plasma concentration versus time curve (AUC) observed at the NOAEL doses cited above, as well as the animal to human exposure ratios (ER) for dexanabinol of high enantiomeric purity, expressed as the ratio of the pharmacokinetic (PK) parameter to that observed for a 150 mg dose in a Phase I study in human volunteers and in Phase II study in patients suffering from severe traumatic brain injury (TBI).

TABLE 6
PK and ER across species, following single i.v. administration of dexanabinol.

| | | Cmax | Ratio to | Ratio to | AUC_{∞} | Ratio to |
|---|---|---|---|---|---|---|
| Study | Dose | (ng/ml) | Phase I | Phase II | (ng × min/ml) | Phase I |
| Rat | 50 mg/kg | | | | | |
| Male | | 102,076 | 20.4 | 51.9 | 4,049,849 | 21.0 |
| Female | | 38,774 | 7.7 | 19.7 | 10,391,118 | 54.0 |
| Rabbit | 25 mg/kg | | | | | |
| Male | | 33,797 | 6.8 | 17.2 | 1,206,621 | 6.3 |
| Female | | 34,544 | 6.9 | 17.6 | 973,407 | 5.1 |
| Male & | | 33,353 | 6.7 | 17.0 | 1,090,290 | 5.7 |
| Female | | | | | | |
| Monkey | 50 mg/kg | | | | | |
| Male | | 166,992 ± 20,569 | 33.4 | 84.9 | 7,040,626 ± 1,785,043 | 36.6 |
| Female | | 175,029 ± 38,333 | 35.0 | 89.0 | 8,169,666 ± 2,867,106 | 42.4 |
| Male & | | 171,010 ± 17,911 | 34.2 | 86.9 | 7,605,146 ± 1,416,806 | 39.5 |
| Female | | | | | | |
| Human | 150 mg | 5,006 ± 434 | 1.0 | NA | 192,547 ± 9,283 | 1.0 |
| Phase I | | | | | | |
| Human | 150 mg | 1,967 ± 253 | NA | 1.0 | 89,019 ± 8,320 | NA |
| Phase II | | | | | | |

The details regarding the human Phase I and Phase II studies have been described (Brewster M. E. et al., International Journal Of Clinical Pharmacology and Therapeutics 35: 361-5, 1997; Knoller N. et al., Crit. Care Med. 30: 548-54, 2002). The results are included in Table 6 for the sake of comparison. In animals, it did appear that clearance was faster in male rats than in females; however, this observation was not replicated in rabbits or monkeys. Both Cmax and AUC in rats, rabbits, and monkeys administered a single dose of dexanabinol at the above NOAELs were well above those observed in the clinical studies.

In 14-day multiple dose pharmacokinetic studies, the NOAEL was 15 mg/kg/day in rats and 25 mg/kg/day in rabbits. In a 28-day study in monkeys the NOAEL was 25 mg/kg/day. Cmax and AUC observed following the last dose at the NOAEL in the multiple dose toxicity studies and the ratios of these values to those observed in the Phase I and II studies for the 150 mg dose are shown in Table 7. Exposure levels as exhibited by the AUC_{∞} associated with the NOAEL in the 14-day studies and 28-day study far exceed those observed in the clinical studies.

TABLE 7
PK and ER across species, following final dose in multiple i.v. administration of
dexanabinol.

| | Dose/ | Cmax | Ratio to | Ratio to | AUC_{∞} | Ratio to | Ratio to |
|---|---|---|---|---|---|---|---|
| Study | Day | (ng/ml) | Phase I | Phase II | (ng × min/ml) | Phase I | Phase II |
| Rat | 15 mg/kg | | | | | | |
| | Day 14 | | | | | | |
| Male | | 30,221 | 6.0 | 15.4 | 1,962,884 | 10.2 | 22.1 |
| Female | | 17,396 | 3.5 | 8.8 | 3,516,133 | 18.3 | 39.5 |
| Rabbit | 25 mg/kg | | | | | | |
| | Day 14 | | | | | | |
| Male | | 149,556 | 29.9 | 76.0 | 4,061,193 | 21.1 | 45.6 |
| Female | | 115,281 | 23.0 | 58.6 | 4,076,845 | 21.2 | 45.8 |
| Male & | | 132,117 | 26.4 | 67.2 | 4,059,030 | 21.1 | 45.6 |
| Female | | | | | | | |
| Monkey | 25 mg/kg | | | | | | |
| | Day 28 | | | | | | |
| Male | | 163,346 ± 13,994 | 32.6 | 83.0 | 12,497,536 ± 1,587,369 | 64.9 | 140.4 |
| Female | | 161,172 ± 6,403 | 32.2 | 81.9 | 10,679,654 ± 249,177 | 55.5 | 120.0 |
| Male & | | 162,259 ± 7,136 | 32.4 | 82.5 | 11,588,595 ± 819,313 | 60.2 | 130.2 |
| Female | | | | | | | |

The NOAELs compared above are based upon 2 weeks and 4 weeks of daily dosing whereas the anticipated clinical regimen consists of a single dose. It is, therefore, reasonable to assume that the NOAELs defined in the multiple-dose animal studies represent an even greater multiple of the human dose if cumulative exposure is considered.

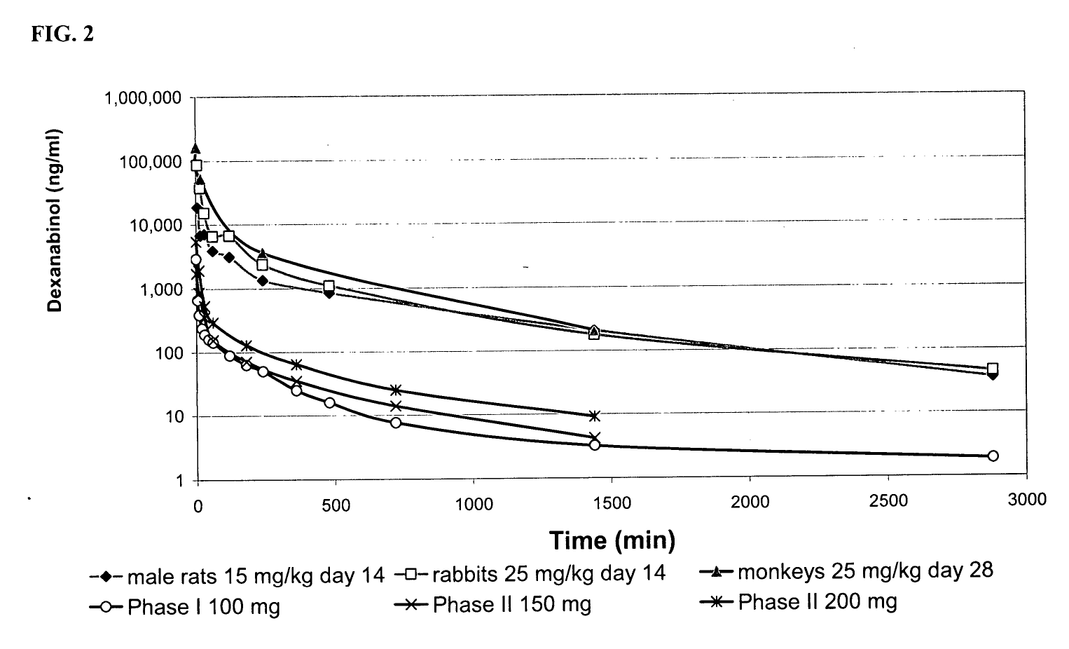

The plasma concentration versus time profiles following the final administration at the NOAEL dose levels in the repeat dose studies in animals are shown in FIG. 2 along with the profile obtained in humans from the Phase I and Phase II studies, that will be described below. The pharmacokinetic profile in all species demonstrated an initial rapid decrease in plasma related concentrations, a common characteristic of highly lipophilic compounds, followed by a slower decline. Plasma concentrations were still detectable, but low, 24 hours after injection, suggesting there might be some accumulation in the repeated dose studies. While there was some evidence for accumulation in the plasma with repeated dosing, the extent of accumulation was minimal.

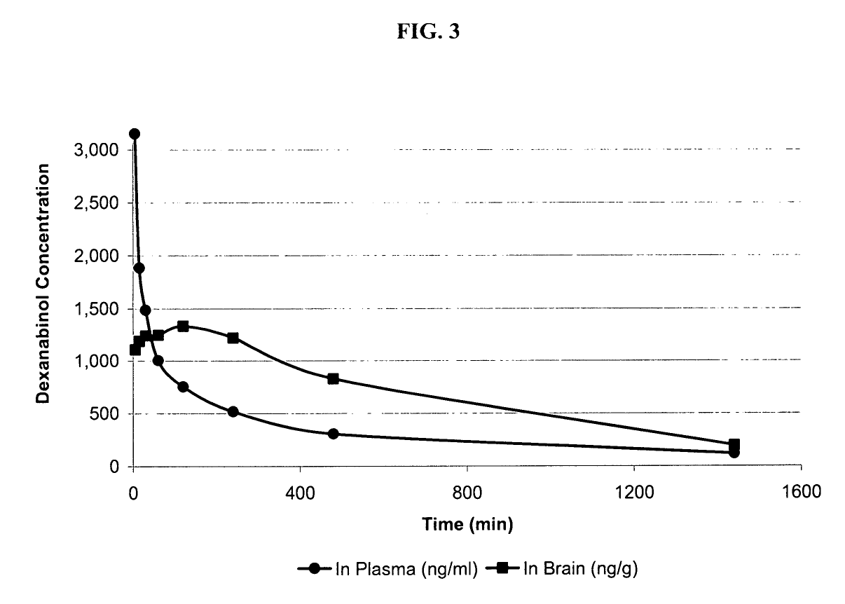

The target organ for dexanabinol therapeutic intervention in patients suffering from TBI being the brain, the monitoring of dexanabinol level in the brain was included in the rat study. Sprague Dawley rats of each sex received a bolus intravenous injection of 4 mg/kg of dexanabinol of high enantiomeric purity in the CREMOPHOR EL®:ethanol clinical formulation. The animals were divided into eight sub-groups of 6 animals, 3 male and 3 female, assigned to a single bleeding time point. The eight bleeding time points were 5, 15, 30 minutes and 1, 2, 4, 8 and 24 hours after injection. Following bleeding the animals were euthanized and their brain were removed for analysis of brain dexanabinol concentrations. The mean rat plasma or brain concentrations of dexanabinol differed between male and female, thus the pharmacokinetic parameters were calculated separately for each gender. The divergences between genders were more pronounced in plasma, reaching 2-3 fold differences for some pharmacokinetic parameters, than in brain, where the differences are not statistically significant for most time points. The results for male and female were averaged in order to compare the levels of dexanabinol in plasma versus brain, following single injection of 4 mg/kg dexanabinol. The results are depicted in FIG. 3. Unlike plasma concentrations, which peaked at the earliest measured time point and rapidly decline in the initial phase, the brain concentrations equilibrated with plasma concentration about 30 minutes after injection. Brain level of dexanabinol continued to increase and displayed a broader peak until levels slowly declined.

Taken together these studies show that the CREMOPHOR EL®:ethanol clinical formulation is efficient for the safe delivery of dexanabinol both into plasma and into the target organ, the brain. The use of dexanabinol of high enantiomeric purity at dose tested caused no psychomimetic side effect in any of the animal species tested.

Human Pharmacokinetic Studies

Following the above-described studies in animals, which demonstrated the safety and the pharmacokinetic profile of the drug, dexanabinol of high enantiomeric purity was then administered to human subjects. According to standard regulatory procedures dexanabinol was first tested in healthy subjects during two Phase I studies, and once its safety was confirmed in humans it was administered to traumatic brain injury patients during a Phase II clinical study.

A Phase I, open label, single center study was conducted to evaluate the safety and tolerance of dexanabinol of high enantiomeric purity following a single intravenous administration to normal, healthy male volunteers (Brewster M. E. et al., International Journal Of Clinical Pharmacology and Therapeutics 35: 361-5, 1997). The trial was designed as a rising-dose tolerance study in healthy young male volunteers. In the study, seven groups of at least six subjects each (in the 100 mg dose n=9) received increasing doses (4, 8, 16, 32, 48, 100, 200 mg/volunteer) of dexanabinol. An additional group of 6 subjects received the vehicle alone. The lower doses, from 4 to 32 mg/volunteer, were included only in the safety segment of the study. Volunteers were followed-up up to 6 days after drug dosing for safety evaluation. Drug administration was well tolerated with no medically important drug-related findings. The conclusions of this safety study were that dexanabinol administered acutely at doses up to and including 200 mg per subject was safe and did not lead to any substantial discomfort to treated subjects.

The pharmacokinetic segment of this study involved 27 healthy male subjects to assess the pharmacokinetic profile of 48, 100 and 200 mg i.v. doses of dexanabinol. Each dosing group, including vehicle control, consisted of 6 healthy male subjects, except for the 100 mg dosing group which contained nine subjects. On the pre-study day, volunteers were treated with 20 mg dexamethasone orally. On the study day, each group was premedicated with Chlorpheniramine maleate 10 mg (H₁ blocker) and Cimetidine 300 mg (H₂ blocker) intravenously, followed 30 minutes later by a single intravenous infusion of dexanabinol, administered using an Ivac peristaltic pump at a rate of 6 ml/min (approximately 15 min infusion/dose). Ten milliliters of blood were then removed (from the contralateral arm) at the end of the infusion, at 5, 10, 20, 30, 45 min, and at 1, 2, 3, 6, 12 and 24 hr post infusion. In some cases blood was also drawn 48 hr post end of infusion.

Mean plasma dexanabinol concentrations, as determined by validated GC/MS/MS analysis, show that for all dose levels, there was an initial rapid decline in plasma concentration followed by a progressively slower decline. Dexanabinol manifested a rapid distributional phase half-life of 2-3 minutes, an intermediate phase elimination half-life of 1-2 hours and a terminal elimination phase half-life of 8.5-9.5 hours. Mean pharmacokinetic parameters were estimated by non-compartmental methods up to 24 hr post-infusion for each dose group (mean±SE; n=6 or 9) using WinNonlin Professional version 3.2, (Pharsight Corp., Mountain view Calif.) and are presented in Table 8.

TABLE 8
Pharmacokinetic analysis of dexanabinol in healthy volunteers (1^{st} Phase I study).

| | | | | AUC_{∞}/ | | | | |
|---|---|---|---|---|---|---|---|---|
| | | AUC_{z} | AUC_{∞} | Dose | | | | |
| Dose | Cmax | (ng × min/ | (ng × min/ | (ng × min/ | CL | t_{1/2} | MRT | Vₛₛ |
| (mg) | (ng/ml) | ml) | ml) | ml) | (ml/min/kg) | (hr) | (hr) | (l/kg) |
| 48 | 1,856 ± 371 | 42,610 ± 4,410 | 43,479 ± 4,453 | 72,469 ± 8,774 | 14.9 ± 1.9 | 6.3 ± 0.7 | 3.1 ± 0.2 | 2.8 ± 0.4 |
| 100 | 2,891 ± 434 | 71,831 ± 5,789 | 73,281 ± 5,773 | 59,374 ± 4,742 | 17.8 ± 1.5 | 6.1 ± 0.7 | 3.1 ± 0.3 | 3.4 ± 0.4 |
| 200 | 4,572 ± 737 | 134,204 ± 6,660 | 139,207 ± 6,131 | 48,162 ± 3,355 | 21.3 ± 1.6 | 8.2 ± 0.7 | 4.0 ± 0.6 | 5.0 ± 0.7 |

Plasma concentrations were approximately 1.8 μg/ml after a 48 mg dose (0.62 mg/kg), 2.9 μg/ml at 100 mg (1.29 mg/kg), and 4.6 μg/ml at 200 mg (2.59 mg/kg). The total areas under the plasma concentration curve (AUC_{∞}) differed significantly for each dose group and were related to the dose in a linear fashion. Total plasma clearance (CL) values of dexanabinol and Vss values increased with the dose, and the AUC_{∞} values normalized for dose (AUC_{∞}/Dose) decreased with the dose. Since CL, Vss are by definition dose-dependent pharmacokinetic parameters (CL=Dose/AUC_{∞} and Vₛₛ=MRT×CL), their increase with dose elevation could be explained by some under-dosing for the higher doses that will result in overestimation of CL and Vₛₛ and under estimation of AUC_{∞}/Dose. Simulations of representative dosing solution preparation by dilutions of dexanabinol concentrate (50 mg/ml), indicated that the 100 mg group was under-dosed by approximately 10% and the 200 mg group was under-dosed by approximately 20%. Compensating for this under-dosing, the overestimated values of CL and Vₛₛ are reduced to statistically non-significant differences across the dose groups (p>0.5 for CL and p>0.2 for Vₛₛ), and AUC_{∞}/Dose underestimated values increase to statistically non-significant differences across the dose groups (p>0.5).

A second human Phase I study involving 24 healthy male volunteers was carried out to compare the pharmacokinetics of dexanabinol following a single i.v. dose of 48 mg or 150 mg. The subjects were divided into two groups of 12 subjects each. Each group was premedicated with ATOSIL® 25 mg (Promethazine H1 blocker), and ZANTAC® 50 mg (Ranitidine H2 blocker) intravenously, followed 15 minutes later by a single short intravenous infusion of dexanabinol lasting 15 minutes. Blood samples for pharmacokinetic assays for dexanabinol were collected immediately before premedication (T=0), immediately after dosing (T=0+) (at the end of the infusion), at 5, 10, 20, 30, 45, and 60 min, and 2, 4, 6, 8, 12, 16, 24, 48, 72 and 96 hr post-dosing. Blood was also collected from four subjects from group 2 (150 mg/kg) on Days 6, 10 and 14. For both dose levels, initial rapid declines in plasma concentration followed by a progressively slower decline were observed.

Pharmacokinetic parameters were estimated by non-compartmental methods up to 96 hr post-infusion, using WinNonlin Professional version 3.2 (Pharsight Corp., Mountain View, Calif., USA). The estimated pharmacokinetic parameters (mean±SE) are shown in Table 9.

TABLE 9
Pharmacokinetic analysis of dexanabinol in healthy volunteers (2^{nd} Phase I study).

| | | | | AUC_{∞}/ | | | | |
|---|---|---|---|---|---|---|---|---|
| | | AUC_{z} | AUC_{∞} | Dose | | | | |
| Dose | Cmax | (ng × min/ | (ng × min/ | (ng × min/ | CL | t_{1/2} | MRT | Vₛₛ |
| (mg) | (ng/ml) | ml) | ml) | ml) | (ml/min/kg) | (hr) | (hr) | (l/kg) |
| 48 | 1,226 ± 118 | 48,673 ± 2,361 | 49,467 ± 2,399 | 78,137 ± 4,305 | 13.2 ± 0.7 | 31.2 ± 3.6 | 8.4 ± 0.5 | 6.6 ± 0.5 |
| 150 | 5,006 ± 434 | 190,806 ± 9,173 | 192,547 ± 9,283 | 93,408 ± 4,224 | 10.9 ± 0.5 | 23.4 ± 1.8 | 6.9 ± 0.4 | 4.5 ± 0.4 |

Intravenous administration of dexanabinol generated high initial plasma levels of the drug (as reflected by values obtained at the end of the drug infusion) that were dose-related.

Maximum plasma concentrations (Cₘₐₓ) were 1.23 μg/ml after the 48 mg dose (0.63 mg/kg), and 5 μg/ml at 150 mg (2.05 mg/kg). In both cases, the drug levels fell rapidly as a function of time with 30 min values being about 11% of the end of infusion levels. The total areas under the plasma concentration curve (AUC) differed significantly for each dose group and increased proportionally to the dose. Total plasma clearance (CL) values of dexanabinol were similar for both dose groups and averaged 12 ml/min/kg across the two dose groups. While pharmacologically, dexanabinol bears little resemblance to naturally occurring cannabinoids, its pharmacokinetic properties are similar to those of Δ⁹-THC and related materials. These properties include rapid initial distribution, long terminal elimination half-life, a rapid total plasma clearance and a large volume of distribution. Altogether, these parameters ensure extensive uptake of the drug into tissues, including the brain and central nervous system, and rapid manifestation of biological action. Beside gathering the pharmacokinetic parameters above described, the two phase I studies allowed to determine that using dexanabinol of high enantiomeric purity in human subjects was safe, well tolerated and no psychomimetic side effects were detected.

The acute nature of events in traumatic brain injury defines a relatively narrow time window for medical intervention, making it scientifically reasonable to assume that the attainment of high peak plasma levels of the drug (Cₘₐₓ) as soon as possible is essential for achieving the high brain drug concentrations necessary for optimal therapeutic activity. Moreover, since dexanabinol is a very lipophilic compound (log P of 7.44), it will cross the blood-brain barrier easily by diffusion, thereby, brain and plasma concentrations will tend to equilibrate fairly quickly. Therefore, higher blood levels will translate into higher brain levels more readily than if some active transport process was involved or if the process was slow and required a long duration of high plasma concentrations. In addition, since dexanabinol is a non-competitive antagonist of the NMDA receptor the faster the administration, the quicker the receptors are saturated and the sooner the pharmacological effect is established.

A Phase II, double masked, multi-center study was conducted to evaluate the safety and tolerance of dexanabinol of high enantiomeric purity following a single intravenous administration in patients with severe head trauma (Knoller N. et al., Crit. Care Med. 30: 548-54, 2002). Treatment was administered within 6 hours of injury, based on the therapeutic window observed in relevant animal models. Additional objectives of the study were to evaluate the long-term outcome of the patients and to determine the optimal dose for Phase III studies.

Medical information was collected en route or upon arrival at the hospital to determine a patient's suitability for enrollment and a randomized patient number was assigned. Written informed consent was obtained from relatives, all eligible patients being in coma. Antihistamines (promethazine hydrochloride PHENERGAN® 25 mg and cimetidine 50 mg) were administered by intravenous bolus injection 15 minutes prior to study drug administration. Dexanabinol was manufactured and formulated as previously described (50 mg/ml in CREMOPHOR EL®:ethanol clinical formulation) and diluted into 100 ml saline prior to injection. Solutions of dexanabinol were infused intravenously using a peristaltic pump (Ivac) at a rate of 6 ml/min (or approximately 15 min/dose). The total doses of dexanabinol scheduled to be administered were 48, 150, or 200 mg per patient. Five milliliters of blood were removed (from the contralateral arm) at the end of the infusion, at 10 and 30 min, and at 1, 3, 6, 12, and 24 hours thereafter for determination of plasma dexanabinol concentrations. Pharmacokinetic parameters were estimated by non-compartmental methods (WinNonlin Professional version 3.2) up to 24 hr post-injection. The estimated pharmacokinetic parameters (mean±SE) are presented in Table 10.

TABLE 10
Pharmacokinetic analysis of dexanabinol in severe TBI patients (Phase II study).

| | | | | AUC_{∞}/ | | | | |
|---|---|---|---|---|---|---|---|---|
| | | AUC_{z} | AUC_{∞} | Dose | | | | |
| Dose | Cmax | (ng × min/ | (ng × min/ | (ng × min/ | CL | t_{1/2} | MRT | Vₛₛ |
| (mg) | (ng/ml) | ml) | ml) | ml) | (ml/min/kg) | (hr) | (hr) | (l/kg) |
| 48 | 1,150 ± 226 | 32,889 ± 4,294 | 34,341 ± 4,383 | 51,846 ± 5,526 | 22.6 ± 3.9 | 7.0 ± 0.9 | 4.1 ± 0.7 | 6.6 ± 2.6 |
| (n = 10) | | | | | | | | |
| 150 | 1,967 ± 253 | 87,166 ± 8,301 | 89,019 ± 8,320 | 43,542 ± 4,329 | 31.0 ± 5.8 | 5.3 ± 0.5 | 3.8 ± 0.4 | 7.5 ± 1.5 |
| (n = 20) | | | | | | | | |
| 200 | 5,793 ± 835 | 190,449 ± 14,268 | 195,116 ± 14,381 | 69,007 ± 5,835 | 17.6 ± 1.9 | 6.3 ± 0.3 | 3.5 ± 0.3 | 4.2 ± 0.9 |
| (n = 21) | | | | | | | | |

Pharmacokinetic parameters are generally dose proportional. Cmax is somewhat lower, and the dose-dependent pharmacokinetic parameters clearance (CL) and volume of distribution at steady-state (Vₛₛ) are somewhat higher, at the 150 mg dose level than would be expected based on the values obtained at the higher and lower doses. This is most likely the result of under-dosing of the 150 mg group. Under-dosing would also lead to under-estimation of the dose-normalized AUC value (AUC/D) which indeed is lower for the 150 mg dose than for the low and high doses. Simulations of the dosing solution preparation indicated that the 150 mg group was under-dosed by approximately 20% while the 48 and 200 mg groups were within 10% of the target dose. Compensating for this under-dosing would reduce the estimated values of CL and Vₛₛ by approximately 20% in the mid dose group. The pharmacokinetic profiles for the three doses, tested in the phase II study are similar to those obtained in the previous phase I studies, specifically there is an initial rapid decrease followed by a slower decline in plasma concentrations.

The primary goal of this study was to establish the safety of dexanabinol of high enantiomeric purity in severe head trauma patients, and indeed dexanabinol was safe and well tolerated in severe head injury. Primary end points included intracranial pressure (ICP), cardiovascular function (heart rate, mean arterial blood pressure, cerebral perfusion pressure and electrocardiogram), clinical laboratory tests, and adverse medical events. The clinical outcome was assessed by the Glasgow outcome scale throughout a six month follow-up period. The nature and incidence of adverse medical events were similar in all groups supporting the safety of dexanabinol of high enantiomeric purity. Moreover, the treated patients achieved significantly better intracranial pressure/cerebral perfusion pressure control without jeopardizing blood pressure. A trend toward faster and better neurological outcome was also observed. Dexanabinol is currently being tested in a Phase III clinical trial for TBI.

It will be appreciated by the skilled artisan that in victims of traumatic brain injury, hypotension is one of the most severe complications and must be avoided. Therefore, it is essential that the drug being administered be free from any contaminant capable of inducing this adverse side effect in this clinical setting. According to the present invention, it has now become feasible to provide dexanabinol at hitherto unobtainable degrees of enantiomeric purity.

As of this date, more than 500 patients have been exposed in international clinical trials to dexanabinol of high enantiomeric purity, and no serious adverse reactions were reported demonstrating the clinical safety of dexanabinol CREMOPHOR EL®:ethanol product without any psychotropic activity or cannabinomimetic adverse effects. A Safety Committee appointed to monitor patient data to ensure patient safety analyzed patients' data after the enrollment and in all cases the safety committee found the drug safe.

### Example 7

### Other Routes of Delivery for Dexanabinol of High Enantiomeric Purity

The route of delivery chosen in the clinical studies was the intravenous route, which is appropriate for rapid drug delivery to the systemic circulation and to target organs in hospital setting in case of acute indications such as TBI. While the preferred route of delivery described for the CREMOPHOR EL®:ethanol clinical formulation is intravenous (i.v.), it is possible to use this formulation for intraperitoneal (i.p.), intramuscular (i.m.), subcutaneous (s.c.), intra cerebro ventricular (i.c.v.), intrathecal and per os (p.o.) administration. For chronic indications other routes can also be used for the delivery of dexanabinol of high enantiomeric purity. These additional routes of administration will also demonstrate the feasibility of further formulations to efficiently deliver dexanabinol.

Oral Delivery

Dexanabinol of high enantiomeric purity (lot # AC9001HU) filled in hard gelatin capsules have shown good oral bioavailability. A pharmacokinetic study for oral bioavailability of dexanabinol in large animals using the minipig model (the best animal model for oral absorption of drugs) was carried out. The animals (n=3) after 8 hours of food deprivation were administered orally with hard gelatin capsules containing dexanabinol of high enantiomeric purity at a dose of 40 mg/kg. Dexanabinol plasma levels were determined using a validated GC-MS assay up to 48 hours following administration. The results obtained showed about 12% oral bioavailability of dexanabinol compared to i.v. injection. Pharmacokinetic analysis was done using the non-compartmental model analysis (WinNonlin software). Linear trapezoidal rule was used to compute AUC and AUMC. The pharmacokinetic parameters for dexanabinol after oral administration are summarized in Table 11.

TABLE 11
PK parameters of dexanabinol after oral administration.

| Cmax | t_{1/2} | AUC* | MRT | AUC/D | |
|---|---|---|---|---|---|
| (ng/ml) | (hr) | (ng * min/ml) | (hr)* | (mg × min/ml) | % F |
| 271.11 ± 101.84 | 7.47 ± 1.06 | 252,568.91 ± 72,387.35 | 12.06 ± 1.15 | 6,445.13 ± 1,846.93 | 12.13 ± 3.48 |

*Total body clearance for extravascular administration.

Rectal Delivery

The neuroprotectant drug dexanabinol was shown to have additional potent anti-inflammatory activity in several animal models. Dexanabinol has demonstrated beneficial effect in a murine model of inflammatory bowel disease (IBD). In chronic gastrointestinal (GI) diseases like IBD, ulcerative colitis or Crohn's disease where gastrointestinal damage exists, the rectal route is preferred for drug administration to avoid adverse effects and additional Gl disturbances and to affect locally the seat of disease. A rectal formulation of enantiomerically pure dexanabinol, which can be administered either as an enema or suppository dosage forms, was developed. The composition of dexanabinol rectal formulation is shown in Table 12.

TABLE 12
Composition of dexanabinol rectal formulation.

| Ingredient | % w/w |
|---|---|
| Dexanabinol of high enantiomeric purity | 0.5% |
| Xanthan gum (KELTROL ® TF) | 1.0% |
| Polyethyleneglycol 1000 (PEG 1000) | 98.5% |

PEG 1000 is an excipient extensively used in rectal preparations and suppository bases. Xanthan gum is a high molecular weight, high viscosity polysaccharide particularly suitable for controlled release applications. The unique solution properties of xantham gum provide many attractive features to pharmaceutical formulations to suspend and stabilize dispersions of solids and immiscible liquids in aqueous systems. Xantham gum provides excellent suspension and thickening properties at very low concentrations. It hydrates well in both acid and alkaline media and the viscosity is relatively unaffected by pH. KELTROL® TF dissolves in cold water at moderate concentrations to produce solutions of high viscosity. The high viscosity is often useful for providing bioadhesion to mucosal surfaces. Bioadhesion to mucous membranes may also be improved by combining xantham gum with polyols. Thus, the combination of xantham gum with PEG 1000 provides an excellent vehicle for bioadhesion and sustained release of the active ingredient dexanabinol increasing its activity and prolonging its residence time in the site of action in the proximity of GI mucosal surfaces.

The PEG 1000 vehicle (USP/NF grade, Spectrum Quality Products, Inc.) was melted in a water bath at ˜50° C. Dexanabinol of high enantiomeric purity was added to the melted PEG 1000 and mixed at 50° C. for about 2 hours until complete dispersion. The xantham gum (KELTROL® TF, Monsanto Pharmaceutical Ingredients) was then added and the mixture was shaken for another 1 hr at 50° C. until a homogeneous dispersion is obtained. The final product melts between 36-38° C., therefore it can be molded to obtain suppositories or it can be administered to animals rectally as an enema by melting it at 40° C. to get a fluid and using a rectal catheter.

Previous animal studies using a murine IBD model demonstrated beneficial effect of dexanabinol administered intra peritoneally. The pharmacological activity of dexanabinol of high enantiomeric purity in enema formulation was also tested and compared to oral and i.p. formulations, wherein oral delivery was performed using a hard gelatin capsule and i.p. delivery was performed using the CREMOPHOR EL®:ethanol formulation. IBD was induced in Sprague Dawley male rats, by rectal administration of a 5% acetic acid solution. The rectum was then washed with saline and animals were clinically observed (body weight, stool consistency and blood in stool) daily for 7 days. On the eighth day, the animals were sacrificed, their colon opened and gross pathology lesions were recorded (hyperemia, edema, number of erosions, ulcers, perforations and adhesions). Animals, at least six per group, were treated rectally (by enema) with either dexanabinol (5, 10, 20 or 40 mg/kg/day) or its vehicle or by oral gavage (20, 40, or 80 mg/kg/day or vehicle). Administration of acetic acid caused a decrease in body weight (up to 20%), changes in stool consistency (diarrhea) and appearance of blood in the stool 24 hours later. Spontaneous clinical healing was detected as animals regained weight and blood was no more evident in the stool. In the gross pathology scale, rectal dexanabinol 10 mg/kg had the best effect (more than 50% reduction of score) compared to its vehicle (p<0.05). Dexanabinol 10 mg/kg reduced also the clinical disease severity compared to the vehicle. The rectal dexanabinol dose of 10 mg/kg was pharmacologically equivalent to 20 mg/kg i.p. dose and 80 mg/kg oral dose. The results of the present work demonstrate the beneficial effects of dexanabinol enema formulation in IBD murine model.

Topical Delivery

The ocular hypotensive effect of cannabinoids intrigued clinicians and investigators alike to exploit these compounds as anti-glaucoma drugs. Nevertheless, the psychotropic effects of these compounds prohibited such large-scale attempts. Ocular hypotensive effect in the rabbit is sometimes difficult to detect due to large intrinsic variations of intra-ocular pressure (IOP) irrespective of the investigated drug. Rabbits were acclimatized for at least a week prior to experiments in the animal facility and their IOP were measured repeatedly prior to data collection. Taking these precautions it was possible to repeatedly demonstrate IOP lowering effect of dexanabinol of high enantiomeric purity upon topical administration in normotensive rabbits.

Dexanabinol was formulated either in submicron emulsion (SME) or in hydroxypropyl-cyclodextrin (HPCD). The solutions of dexanabinol in HPCD were prepared as follows. First, a weighted amount of dexanabinol was dissolved in a minimum amount of absolute ethanol. The drug containing ethanol solution is then added dropwise to the HPCD powder, which is subsequently dried at 48-80° C. until ethanol evaporates. Water is then added and mixed with the dried powder to give final dexanabinol concentrations of 0.1 to 2 mg/ml and HPCD concentrations of 5 to 45%. Complete dissolution is obtained by sonication and heating. The homogenous solutions are then filtered through 0.2-0.45μm sterile disposable filter unit.

TABLE 13
Dexanabinol formulation in SME.

| Phase | Ingredient | % w/v |
|---|---|---|
| Oil | MCT Oil | 4.25% |
| | Lecithin (LIPOID E-80 ®) | 0.75% |
| | DL-α-Tocopherol Succinate | 0.02% |
| | Dexanabinol | 0.10% |
| Water | Polysorbate 80 | 1.00% |
| | Glycerol | to 300 mOsm (˜2.25%) |
| | EDTA | 0.10% |
| | Benzalkonium Chloride | 0.01% |
| | Purified water | to 100% |

Submicron emulsions are made of homogenous oily droplets in a size range of 50-80 nm emulsified in aqueous solution. Various ocular drugs, including timolol, pilocarpine and indomethacin were successfully formulated in SME and were advantageous over the standard fomulations both in terms of irritation and bioavailability. The composition of dexanabinol topical formulation is shown in Table 13.

A total volume of about 100 ml (100 g w/w % ) of dexanabinol in SME was prepared. The oil phase stock was composed of Medium-chain triglyceride (MCT) Oil, LIPOID E-80® and DL-α-Tocopherol Succinate and dexanabinol of high enantiomeric purity. The lipids and oil were weighed in a 250 ml beaker and mixed at 40-45° C. using a magnetic stirrer for 15 min until a homogenous and almost clear solution was obtained. Dexanabinol was then dissolved in the oil phase by stirring at room temperature (RT). The water phase was prepared as follows. Polysorbate 80, EDTA disodium, glycerol, and benzalkonium chloride were dissolved at RT in purified water up to a final weight of 100 g in 250 ml beaker by gentle shaking using the magnetic stirrer plate until a clear homogenous solution was obtained. The gentle stirring is aimed to avoid the formation of bubbles in the solution. Each material is dissolved in the water separately, in the specified order of addition.

Once both phases are ready they are mixed according to the following procedure. Oil Phase (5 g) was heated to 40-45° C. and added to the beaker containing the water phase (preheated to 40-45° C.). The mixture was gently stirred for 10-15 minutes at room temperature. First a coarse Oil-in-Water emulsion is prepared, using the medium-sized dispenser and homogenizing unit Polytron PT3000 at 12,000 rpm for 3 minutes. The temperature during the Polytron step should be in the range of 25-45° C. The resultant micron size emulsion was cooled at room temperature. The droplet size of the emulsion obtained after Polytron step was lowered to the submicron (nanosize) range by submitting the emulsion to high shear homogenization using the Gaulin Microlab 70 or Emulsiflex High Pressure Homogenizers at 800 bar pressure. A total of 3 to 6 cycles were performed to obtain homogeneous SME preparation with a mean droplet diameter in the range of 50-100 nm. The pH of the resultant SME was adjusted to 7.4 by adding small amounts of 1 N HCl or 1 N NaOH solutions using a calibrated pH meter. The osmolalities of all SME obtained were around 300 mOsm. If the value obtained is below 300±30 mOsm it must be adjusted by adding Glycerol. The SME formulations were sterilized by filtration through a 0.2 μm sterile disposable filter unit (cellulose acetate, 0.5 liter volume, Corning, England), using vacuum supplied by water pump. The SME formulations were packaged under aseptic conditions in 5 ml plastic droppers in laminar flow hood using sterile (by gamma irradiation) low density polyethylene (LDPE) eye drop bottles, insert and caps. This dexanabinol SME formulation was then tested in normotensive rabbits.

New Zealand White albino rabbits weighing 2-2.5 kg were acclimatized in our animal facility for at least a week prior to IOP measurements. Drug, saline and blank-vehicle groups (n=8-12 animals per group) were included. In most experiments, 50 μl drops were applied at 09:00 and measurements were taken 1 hour, 3, 5, and 7 hours later. Baseline IOP was individualized for each animal and time point prior to drug testing. IOP was measured by a Digilab Pneumatonometer, Model 30R. ΔIOP was calculated by subtracting the baseline IOP from IOP value measured after drug application at a corresponding time point. Maximal ΔIOP and area under the ΔIOP×hours curve (AUC) were calculated and averaged for each group. Data of IOP, AUC, blood pressure and toxicity were analyzed by the Wilcoxon-Rank test.

Average values of maximal ΔIOP after topical instillation of dexanabinol of high enantiomeric purity varied between 7.0 mmHg to 1.6 mmHg in several experiments with a range of dosages and formulations. IOP was reduced in dexanabinol treated groups compared to blank-vehicle and saline groups in virtually all experiments. The blank SME vehicle was devoid of any significant IOP lowering activity as compared to saline treatment. In one study, groups of rabbits (n=8, each) were followed for three days with a daily application of dexanabinol of high enantiomeric purity and demonstrated a sustained IOP lowering effect over that time period.

Dose dependency was studied using 0.02%; 0.05%; 0.1%; 0.2% dexanabinol; vehicle and saline control groups in a masked study. A small, yet statistically significant (p<0.05) IOP lowering effect of all doses of dexanabinol of high enantiomeric purity compared to the vehicle and the saline was demonstrated. The most effective dose was 0.1% (AUC=14.6±2.2 mmHg×Hour±SEM; Maximal ΔIOP=2.9±0.4 mmHg). Other dosages of 0.02%; 0.05% and 0.2% were significantly less effective with AUC=6.4±2.5; 5.1±3.7 and 6.9±1.7 mmHg×Hour, respectively and maximal ΔIOP of 1.8±0.7 mmHg; 1.8±0.9; and 2.2±0.6, respectively. Similar AUC values were found by us for other commercially available drugs: Timolol (Merck Sharp & Dohme) and levobunolol HCl (Allergan) yielded 7.3±3.0 and 13.3±1.5 mmHg×Hours respectively.

Ocular toxicity was tested as follows. Two groups of five animals each received topical instillation of dexanabinol in SME and blank SME for five days four times daily. The animals were examined daily with a slit lamp for ocular discharge, conjunctival hyperemia, corneal fluorescein staining and iris hyperemia. Results were scored on a 0.0-4.0 scale in 0.5 steps. Topical treatment with dexanabinol of high enantiomeric purity resulted in mild conjunctival injection and discharge that did not significantly differ from SME-blank treated animals. Corneal staining, corneal opacities and iris hyperemia did not occur in any of the animals.

Dose dependency and a clear effect on aqueous humor dynamics were shown for topical administration. In this model, it was possible to demonstrate that topical application of dexanabinol is non psychotropic and has none of the ocular adverse effects typical to other cannabinoids Our data suggest that these investigations of dexanabinol of high enantiomeric purity for the therapy of glaucoma may yield a new generation of anti-glaucoma drugs with both IOP lowering and neuroprotective effects. The retino-neuroprotective effect of dexanabinol of high enantiomeric purity is currently studied on ischemic retina in rabbits. Such neuroprotective effect may prove to be critically important in preserving ganglion fibers in glaucomatous damaged optic nerves.

Altogether these studies show that dexanabinol of high enantiomeric purity, or its pharmaceutically acceptable salts or esters derivatives, can be prepared in various types of formulations and administered by various routes of administration to treat the diseases induced in the models above-described.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

What is claimed is:

1. A compound of formula (I): 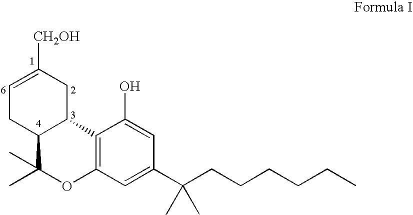
having the (3S,4S) configuration and being in enantiomeric excess of at least 99.90% over the (3R,4R) enantiomer, or a pharmaceutically acceptable salt, ester or solvate of said compound.

2. The compound of claim 1 or a pharmaceutically acceptable salt, ester or solvate of said compound, having the (3S,4S) configuration and being in enantiomeric excess of at least 99.92% over the (3R,4R) enantiomer.

3. The compound of claim 2 or a pharmaceutically acceptable salt, ester or solvate of said compound, having the (3S,4S) configuration and being in enantiomeric excess of at least 99.95% over the (3R,4R) enantiomer.

4. The compound of claim 3 or a pharmaceutically acceptable salt, ester or solvate of said compound, having the (3S,4S) configuration and being in enantiomeric excess of at least 99.97% over the (3R,4R) enantiomer.

5. A compound of formula (I): 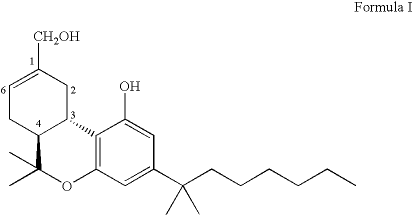
having the (3S,4S) configuration and being in absolute enantiomeric amount of at least 99.95% and wherein the (3R,4R) enantiomer is in absolute enantiomeric amount of 0.05% or less, or a pharmaceutically acceptable salt, ester or solvate of said compound.

6. The compound of claim 5 or a pharmaceutically acceptable salt, ester or solvate of said compound, having the (3S,4S) configuration and being in absolute enantiomeric amount of at least 99.96% and wherein the (3R,4R) enantiomer is in absolute enantiomeric amount of 0.04% or less.

7. The compound of claim 6 or a pharmaceutically acceptable salt, ester or solvate of said compound, having the (3S,4S) configuration and being in absolute enantiomeric amount of at least 99.97% and wherein the (3R,4R) enantiomer is in absolute enantiomeric amount of 0.03% or less.

8. The compound of claim 7 or a pharmaceutically acceptable salt, ester or solvate of said compound, having the (3S,4S) configuration and being in absolute enantiomeric amount of at least 99.98% and wherein the (3R,4R) enantiomer is in absolute enantiomeric amount of 0.02% or less.

9. A pharmaceutical composition comprising as an active ingredient dexanabinol, a compound of formula (I): 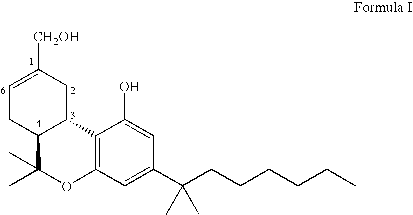
having the (3S,4S) configuration and being in enantiomeric excess of at least 99.90% over the (3R,4R) enantiomer, or a pharmaceutically acceptable salt, ester or solvate of said compound.

10. The pharmaceutical composition according to claim 9 wherein the active ingredient dexanabinol, or a pharmaceutically acceptable salt, ester or solvate of said compound, has the (3S,4S) configuration and is in enantiomeric excess of at least 99.92% over the (3R,4R) enantiomer.

11. The pharmaceutical composition according to claim 10 wherein the active ingredient dexanabinol, or a pharmaceutically acceptable salt, ester or solvate of said compound, has the (3S,4S) configuration and is in enantiomeric excess of at least 99.95% over the (3R,4R) enantiomer.

12. The pharmaceutical composition according to claim 11 wherein the active ingredient dexanabinol, or a pharmaceutically acceptable salt, ester or solvate of said compound, has the (3S,4S) configuration and is in enantiomeric excess of at least 99.97% over the (3R,4R) enantiomer.

13. A pharmaceutical composition comprising as an active ingredient dexanabinol, a compound of formula (I): 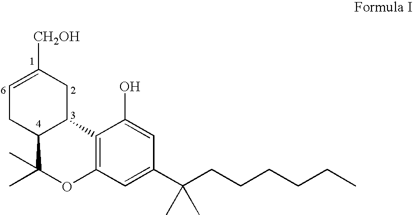
having the (3S,4S) configuration and being in absolute enantiomeric amount of at least 99.95% and wherein the (3R,4R) enantiomer is in absolute enantiomeric amount of 0.05% or less, or a pharmaceutically acceptable salt, ester or solvate of said compound.

14. The pharmaceutical composition according to claim 13 wherein the active ingredient dexanabinol, or a pharmaceutically acceptable salt, ester or solvate of said compound, has the (3S,4S) configuration and is in absolute enantiomeric amount of at least 99.96% and wherein the (3R,4R) enantiomer is in absolute enantiomeric amount of 0.04% or less.

15. The pharmaceutical composition according to claim 14 wherein the active ingredient dexanabinol, or a pharmaceutically acceptable salt, ester or solvate of said compound, has the (3S,4S) configuration and is in absolute enantiomeric amount of at least 99.97% and wherein the (3R,4R) enantiomer is in absolute enantiomeric amount of 0.03% or less.

16. The pharmaceutical composition according to claim 15 wherein the active ingredient dexanabinol, or a pharmaceutically acceptable salt, ester or solvate of said compound, has the (3S,4S) configuration and is in absolute enantiomeric amount of at least 99.98% and wherein the (3R,4R) enantiomer is in absolute enantiomeric amount of 0.02% or less.

17. The pharmaceutical composition according to claim 9 further comprising a pharmaceutically acceptable diluent or carrier.

18. The pharmaceutical composition according to claim 17 wherein the diluent comprises an aqueous cosolvent solution comprising a pharmaceutically acceptable cosolvent, a micellar solution or emulsion prepared with natural or synthetic ionic or non-ionic surfactants, or a combination of such cosolvent and micellar or emulsion solutions.

19. The pharmaceutical composition according to claim 17 wherein the carrier comprises a solution of ethanol, a surfactant and water.

20. The pharmaceutical composition according to claim 17 wherein the carrier is an emulsion comprising triglycerides, lecithin, glycerol, an emulsifier, and water.

21. The pharmaceutical composition according to claim 17 comprising a cosolvent solution comprising polyoxyl 35 castor oil and ethanol.

22. The pharmaceutical composition according to claim 21 wherein the polyoxyl 35 castor oil is present in an amount of 30-80% w/w and the ethanol is present in an amount of 20-70% w/w.

23. The pharmaceutical composition according to claim 22 wherein the polyoxyl 35 castor oil is present in an amount of 45-80% w/w and the ethanol is present in an amount of 20-55% w/w.

24. The pharmaceutical composition according to claim 23 wherein the polyoxyl 35 castor oil is present in an amount of 60-80% w/w and the ethanol is present in an amount of 20-40% w/w.

25. The pharmaceutical composition according to claim 21 further comprising a preservative, an antioxidant or a combination thereof.

26. The pharmaceutical composition according to claim 25 wherein the antioxidant is DL-α-tocopherol optionally supplemented with edetic acid.

27. The pharmaceutical composition according to claim 26 comprising 0.1-5% w/w DL-α-tocopherol and 0.001-0.1% w/w edetic acid.

28. The pharmaceutical composition according to claim 9 in unit dosage form.

29. The pharmaceutical composition according to claim 28 suitable for oral administration.

30. The pharmaceutical composition according to claim 28 suitable for parenteral administration.

31. A method for preventing, alleviating or treating neurological disorders, chronic degenerative diseases, CNS poisoning, cognitive impairment, inflammatory diseases or disorders, autoimmune diseases or disorders, pain, emesis, glaucoma and wasting syndromes, by administering to an individual in need thereof a prophylactically or therapeutically effective amount of a pharmaceutical composition comprising as an active ingredient a compound according to claim 1.

32. The method of claim 31 wherein the compound has an enantiomeric excess of at least 99.92% over the (3R,4R) enantiomer.

33. The method of claim 32 wherein the compound has an enantiomeric excess of at least 99.95% over the (3R,4R) enantiomer.

34. The method of claim 33 wherein the compound has an enantiomeric excess of at least 99.97% over the (3R,4R) enantiomer.

35. The method of claim 34 wherein the compound is administered to an individual to treat a neurological disorder.

36. A method for preventing, alleviating or treating neurological disorders, chronic degenerative diseases, CNS poisoning, cognitive impairment, inflammatory diseases or disorders, autoimmune diseases or disorders, pain, emesis, glaucoma and wasting syndromes, by administering to an individual in need thereof a prophylactically or therapeutically effective amount of a pharmaceutical composition comprising as an active ingredient a compound according to claim 5.

37. The method of claim 36 wherein the compound is in absolute enantiomeric amount of at least 99.96% and wherein the (3R,4R) enantiomer is in absolute enantiomeric amount of 0.04% or less.

38. The method of claim 37 wherein the compound is in absolute enantiomeric amount of at least 99.97% and wherein the (3R,4R) enantiomer is in absolute enantiomeric amount of 0.03% or less.

39. The method of claim 38 wherein the compound is in absolute enantiomeric amount of at least 99.98% and wherein the (3R,4R) enantiomer is in absolute enantiomeric amount of 0.02% or less.

40. The method of claim 39 wherein the compound is administered to an individual to treat a neurological disorder.
